SEQUENCE LISTING (2 sequences)

SEQ ID NO: 1 - protein, 8 aa, Artificial Sequence
  FLAG epitope tag
DYKDDDDK

SEQ ID NO: 2 - protein, 11 aa, Artificial Sequence
  FLAG epitope tag with a linker sequence
GGADYKDDDDK

(19) United States
(12) Patent Application Publication
(10) Pub. No.: US 2010/0247433 A1
(43) Pub. Date: Sep. 30, 2010

(54) Use of non-canonical amino acids as metabolic markers for rapidly-dividing cells

(75) Inventors: David Tirrell, Pasadena, CA (US); Daniela C. Dieterich, Pasadena, CA (US); Aaron J. Link, Austin, TX (US); Erin Schuman, Pasadena, CA (US)

Correspondence Address: ROPES & GRAY LLP, PATENT DOCKETING 39/41, ONE INTERNATIONAL PLACE, BOSTON, MA, 02110-2624, US

(73) Assignee: California Institute of Technology, Pasadena, CA (US)

(21) Appl. No.: 11/581,713

(22) Filed: Oct. 16, 2006

Related U.S. Application Data
(60) Provisional application No. 60/727,076, filed on Oct. 14, 2005.

(51) Int. Cl. A61K 51/04; A61K 49/00; A61K 31/655; A61K 31/4164; A61K 31/56; A61K 31/7076; A61K 31/445; A61K 38/46; G01N 33/53

(52) U.S. Cl. 424/1.65; 424/9.1; 514/151; 514/398; 514/169; 514/46; 514/328; 424/94.6; 435/7.1

(57) ABSTRACT

The invention provides methods, reagents and systems to preferentially mark fast-proliferating cells/tissues (such as cancer), by incorporating non-natural amino acids into proteins, preferably in vivo, using the endogenous protein synthesis machinery of an organism. The incorporated non-natural amino acids contain reactive groups for further chemical reagents, which may serve as a “handle” to for a number of uses, such as imaging of cancer cells, targeting drugs to preferentially kill cancer cells, and proteomic analysis in the context of large scale or high throughput screening for candidate drug leads that affects the proliferation of a target cell, etc.

### REFERENCE TO RELATED APPLICATIONS

This application claims the benefit of the filing date of U.S. Provisional Application U.S. Ser. No. 60/727,076, filed on Oct. 14, 2005. The entire teaching of the referenced application is incorporated herein by reference.

### GOVERNMENT SUPPORT

Work described herein was funded, in whole or in part, by Grant No. DAAD19-03-D-0004 (ARO) from the United States Army. The United States government has certain rights in the invention.

### BACKGROUND OF THE INVENTION

Cells respond to fluctuations in their environment by changing the set of proteins they express. Understanding such changes is not only important for the understanding of cellular processes but also for the understanding of how pharmaceuticals alter such expression patterns. Alterations in protein synthesis and degradation enable cells to adapt to changing external conditions.

Tumors are the result of uncontrolled cell divisions. For years, researchers have been seeking therapies that specifically target the tumor but leave the surrounding normal tissue unaffected. Currently treatments for cancerous tumors include one or more of the following: surgical removal of tumor, radiation of tumors or administration of drugs to halt or suppress tumor growth. A disadvantage of current cancer therapies is their toxic and potentially fatal side effects, owing to the fact that tumor cells can often not be distinguished from normal surrounding tissue.

Accordingly, there is a need for new methods, systems, and reagents for differentiating fast proliferating tissues (such as cancer or other pathological proliferative conditions) from the surrounding normal tissues, for a wide range of uses, such as medical diagnosis and/or treatment.

### SUMMARY OF THE INVENTION

The invention provides methods and reagents for metabolic labeling and detection of rapidly-dividing cells, such as those found in tumors. The methods for labeling and/or detection are partly based on bioorthogonal incorporation of non-natural amino acids into newly synthesized proteins of those rapidly-dividing cells. Following labeling, a reactive group on the non-natural amino acid can be coupled to another custom-designed labeling group/moiety to enable the visualization, irradiation or destruction of the tumor cells.

Thus one aspect of the invention provides a method for detecting or treating cancer in a patient, comprising administering to the patient a pharmaceutical composition comprising: (1) a non-natural amino acid comprising a first reactive group; (2) a labeling reagent comprising a second reactive group and a labeling moiety; under conditions wherein the non-natural amino acid is preferentially incorporated into the newly synthesized proteins of the cancer; wherein the first and second reactive groups react to label the non-natural amino acid with the labeling reagent; wherein the labeling reagent comprises a detectable label, inhibits the progression of the cancer, and/or facilitates the killing of the cancer.

In a related aspect, the invention provides a use of a pharmaceutical composition in the preparation of a medicament for treating cancer in a patient, wherein the pharmaceutical composition comprises: (1) a non-natural amino acid comprising a first reactive group; (2) a labeling reagent comprising a second reactive group and a labeling moiety; under conditions where the non-natural amino acid is preferentially incorporated into the newly synthesized proteins of the cancer; wherein the first and second reactive groups react to label the non-natural amino acid with the labeling reagent; wherein the labeling reagent comprises a detectable label, inhibits the progression of the cancer, and/or facilitates the killing of the cancer.

In one embodiment, the cancer is at least about 50% more preferentially labeled than a surrounding normal tissue.

In one embodiment, the first reactive group is an azido group. For example, in one embodiment, the non-natural amino acid is azidoalanine, azidohomoalanine (AHA), azidonorvaline, or azidonorleucine.

In one embodiment, the first reactive group is a ketone or aldehyde moiety.

In one embodiment, the first reactive group is a diboronic acid moiety.

In one embodiment, the first reactive group is a terminal alkyne moiety, such as HPG.

In one embodiment, the non-natural amino acid is incorporated in vivo by endogenous protein synthesis machinery of the cell or the tissue.

In one embodiment, the non-natural amino acid is site-specifically incorporated in place of a natural amino acid selected from methionine or phenylalanine.

In other embodiments, the non-natural amino acid is site-specifically incorporated in place of any other natural amino acids.

In one embodiment, the labeling reagent comprises a chelate moiety for chelating a metal.

In one embodiment, the labeling reagent is a chelator for a radiometal or a paramagnetic ion.

In one embodiment, the method further comprises infusing into the patient an effective amount of chelator compounds.

In one embodiment, the chelator compound is EDTA or DTPA.

In one embodiment, the labeling reagent is a chelator for a radionuclide useful for radiotherapy or imaging procedures.

In one embodiment, the radionuclide is a beta- or alpha-emitter for radio-therapeutic use.

In one embodiment, the radionuclide is a gamma-emitter, positron-emitter, Auger electron-emitter, X-ray emitter or fluorescence-emitter.

In one embodiment, the radionuclide is ^{99m}Tc (technium).

In one embodiment, the labeling reagent comprises a bifunctional chelator NₓS_{y} that are capable of coordinately binding a metal or radiometal, wherein x and y are integers between 1 and 4.

In one embodiment, the NₓS_{y} has a N₂S₂ or a N₃S core.

In one embodiment, the labeling reagent comprises a cytotoxic moiety.

In one embodiment, the cytotoxic moiety is a radiosensitizing agent, a Boron addend, a chemotherapeutic agent, a protein synthesis inhibitor, a prodrug activated by host metabolism, a cytotoxic toxin, an enzyme that converts prodrug locally, or a dye used in photodynamic therapy or in conjunction with appropriate non-ionizing radiation.

In one embodiment, the radiosensitizing agent is selected from: nitroimidazoles, metronidazole or misonidazole.

In one embodiment, the Boron addend is carborane.

In one embodiment, the chemotherapeutic agent is: taxol; nitrogen mustards; ethylenimine derivatives; alkyl sulfonates; nitrosoureas; triazenes; pyrimidine analogs; purine analogs; vinca alkaloids; antibiotics; enzymes; platinum coordination complexes; substituted urea; methyl hydrazine derivatives; adrenocortical suppressants; or hormones and antagonists selected from: adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), or androgens (testosterone propionate and fluoxymesterone).

In one embodiment, the protein synthesis inhibitor is puromycin, cycloheximide, or ribonuclease.

In one embodiment, the labeling reagent comprises a prodrug that is only activated from its inactive precursor form by host metabolism.

In one embodiment, the cytotoxic toxin is selected from: ricin, ricin A chain (ricin toxin), Pseudomonas exotoxin (PE), diphtheria toxin (DT), Clostridium perfringens phospholipase C(PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin or volkensin.

In one embodiment, the enzyme that converts prodrug locally is alkaline phosphatase, and said prodrug is etoposidephosphate.

In one embodiment, the cytotoxic moiety is administered to the patient at a dose that contain 10-100 times less active agent as an active moiety than the dosage of agent administered as unconjugated active agents.

In one embodiment, the labeling reagent further comprises an antigenic moiety that can be recognized by an antibody. For example, the affinity moiety may be biotin, and the antigenic moiety may be an epitope tag, such as a FLAG tag, an HA tag, a His₆ tag, etc.

In one embodiment, the FLAG tag comprises one or more cleavage sites for a sequence-specific protease, such as trypsin.

In one embodiment, the FLAG tag is situated between the affinity moiety and the second reactive group.

In one embodiment, the second reactive group and the affinity moiety are linked by one or more cleavable functional groups, such as photo-cleavable groups, chemically cleavable groups, or enzymatically cleavable groups.

In one embodiment, the second reactive group and the affinity moiety are linked by one or more cleavage sites for a sequence-specific protease, such as Factor Xa or PreScission Protease.

In one embodiment, the second reactive group and the labeling moiety are linked by a photo-cleavable linker.

In one embodiment, the condition is such that the concentration of the non-natural amino acid is optimized to effect a pre-selected amount or percentage of incorporation.

In one embodiment, the method is carried out by contacting the tissue or the cell cultured in vitro.

In one embodiment, the method is carried out by administering the non-natural amino acid to an animal.

In one embodiment, the cell or the tissue is further contacted with a second non-natural amino acid. The second non-natural amino acid may contain an isotope tag, such as a deuterated natural amino acid. The isotope may be one or more of ²H, ³H, ¹³C, ¹⁴C, ¹⁵N, ¹⁸O, or ³⁵S.

In one embodiment, the method further comprises administering to the patient a second pharmaceutical composition comprising: (3) a second non-natural amino acid comprising a third reactive group; (4) a second labeling reagent comprising a fourth reactive group and a second labeling moiety; under conditions where the second non-natural amino acid is preferentially incorporated into the newly synthesized proteins of the cancer; wherein the third and fourth reactive groups react to label the second non-natural amino acid with the second labeling reagent; wherein at least one of the labeling reagents inhibits the progression and/or facilitates the killing of the cancer.

Another aspect of the invention provide a high throughput screening method for identifying a compound that inhibits cell proliferation, the method comprising: (1) contacting a control cell with a non-natural amino acid comprising a first reactive group; (2) contacting a control cell with a labeling reagent comprising a second reactive group and a labeling moiety; under conditions where the non-natural amino acid is preferentially incorporated into the newly synthesized proteins of the control cell; wherein the first and second reactive groups react to label the non-natural amino acid with the labeling reagent; (3) repeat steps (1) and (2) to label a test cell at the presence of a candidate compound; (4) comparing the quantity of the labeling moiety in the control cell and the test cell, respectively; wherein a decrease in quantity of the labeling moiety in the test cell is indication that the candidate compound inhibits proliferation of the test cell.

In one embodiment, the control cell and the test cell are primary cancer cells from the same cancer, or are from the same cancer cell line.

In one embodiment, the method further comprises testing the general toxicity of the candidate compound on a normal or healthy cell.

In one embodiment, the labeling moiety is a fluorescent moiety or a reagent that can be subsequently coupled to a fluorescent reagent, and wherein step (4) is effectuated by monitoring fluorescent intensity.

Another aspect of the invention provides a method for detecting or imaging cancer in a patient, comprising: (a) contacting the patient with a non-natural amino acid comprising a first reactive group, under conditions where the non-natural amino acid is preferentially incorporated into the newly synthesized proteins of the cancer; (b) contacting the patient with a labeling reagent comprising a second reactive group and a labeling moiety, wherein the first and second reactive groups react to label the non-natural amino acid with the labeling reagent; (c) detecting the labeling moiety, thereby detecting cancer in the patient.

In one embodiment, the labeling moiety comprises an imaging agent.

In one embodiment, the imaging agent is a radionuclide imaging agent.

In one embodiment, the radionuclide imaging agent is radioactive iodine or indium.

In one embodiment, the labeling moiety is detected by radioscintigraphy, magnetic resonance imaging (MRI), computed tomography (CT scan), or positron emission tomography (PET).

In one embodiment, the contacting step (a) or (b) is independently effected by administering to the patient the non-natural amino acid or the labeling reagent orally, perentarally, transdermally, topically, intramuscularly (i.m.), intraperitoneally (i.p.), or intraveneously (i.v.).

In one embodiment, the detecting step (c) includes determining the volume, shape and/or location of the cells labeled by the labeling reagent in the patient.

Another aspect of the invention provides a kit, comprising: (1) one or more non-natural amino acids capable of being incorporated into newly synthesized proteins in a translation system, such as in vivo in an animal, in vitro in tissue culture systems, or in a cell-free in vitro translation system (optionally supplemented with modified tRNA and/or tRNA AARS); and (2) one or more labeling reagents of the invention, such as a subject cancer killing labeling reagent comprising a cancer killing moiety (supra).

In a preferred embodiment, the kit comprises two or more pairs of non-natural amino acids/labeling reagents, where the labeling moieties on the two label reagents are different (for example, different fluorescent markers, drugs or other cancer killing agents, etc.)

The kit of the invention may be provided as a therapeutic kit for treating a patient (e.g., a cancer patient) using the methods of the invention.

The kit of the invention may be provided as a diagnostic kit for detecting cancer or other fast proliferating cells in a patient (e.g., a cancer patient) using the methods of the invention.

The kit of the invention may also be provided as a research kit for in vitro cell labeling using the methods of the invention. In a preferred embodiment, the cell is a neuronal cell, such as cultured primary neurons, neuronal cell lines, or stem/progenitor cells committed to the neuronal differentiation pathway.

The kits of the invention may further comprise an instruction or label for the intended use of the kit.

The embodiments described above, including those described under different aspects of the invention, are contemplated to be applicable for all aspects of the inventions wherever appropriate.

### BRIEF DESCRIPTION OF THE FIGURES

FIG. 1: Overview of the protein identification procedure using non-natural amino acids, such as azidohomoalanine (AHA). Cells are challenged in the presence of AHA to allow for protein synthesis with AHA-incorporation (“+/−” represents the presence/absence of a modulator of activity). After incubation, cells are lysed or undergo a subcellular fractionation for biochemical enrichment of specific cellular compartments followed by lysis. Lysates are then coupled to an alkyne-bearing affinity tag, followed by affinity chromatography to enrich for AHA-incorporated proteins. Purified proteins are digested with a protease, and the resulting peptides are analyzed by tandem mass spectrometry to obtain experimental spectra. Different search programs are used to match the acquired spectra to protein sequences.

FIG. 2A: Generation of a tandem featured alkyne tag for two-step purification of AHA incorporated proteins. Structure of a Biotin-FLAG-alkyne affinity reagent. Biotin (square), alkyne (square circle) as well as the tryptic cleavage sites (scissors) are indicated. The FLAG epitope DYKDDDDK (SEQ ID NO: 1) is separated from the biotin moiety by a short linker (GGA).

FIG. 2B: Western blot analysis for tandem purified biotinylated proteins using the Biotin-FLAG-alkyne tag. Cell lysates from both AHA and methionine incubated HEK293 cells were subjected to [3+2]-cycloaddition with the Biotin-FLAG-alkyne tag and subsequently purified using monomeric avidin and anti-FLAG-M2 affinity matrices. SDS was used in both steps to elute the proteins from the resin. SDS was removed from avidin-eluates by ultrafiltration and neutralization with Triton X-100 before incubation with the FLAG-M2 affinity matrix. Sizes of marker proteins are indicated on the left margin.

FIG. 3A: The method of AHA incorporation in acute hippocampal slices. 400 μm-thick hippocampus slices were isolated from male Sprague-Dawley rats. These slices were recovered in artificial cerebrospinal fluid (ACSF) for at least 1 hour on organotypic membranes. Upon recovery, the slices were incubated with either a mixture of ³⁵S Met and ³⁵S Cys or varying concentrations of AHA for 1.5 hours.

FIG. 3B: Autoradiogram of ³⁵S Met and ³⁵S Cys labeled proteins from acute hippocampal slices. Labeled slices were homogenized and varying volumes of the homogenate are shown here. Newly synthesized proteins within the slices readily acquire the ³⁵S labeled amino acids.

FIG. 3C: Western blot analysis for AHA-incorporated proteins from acute hippocampal slices. After AHA incorporation and slice homogenization, the homogenates were subjected to [3+2]-cycloaddition with a Biotin-PEO tag and concentrated using Neutravidin-conjugated beads. Note the vast biotinylation of proteins due to the incorporation of AHA into the acute hippocampal slices.

FIG. 4: Detection of newly synthesized proteins in cells of acutely dissected rat brain tissue.

FIG. 5: Detection of newly synthesized proteins in cells of acutely dissected human brain tissue. Tumor and control cells were trypsinized and triturated before incubation with 4 mM AHA for 1 hour at 37° C. After cell lysis and Cu(I)-catalyzed tagging with a biotin-FLAG-alkyne tag, samples were subjected to Neutravidin-purification to separate newly synthesized proteins from pre-existing proteins, and analyzed on a Western blot using an anti-biotin antibody. Same total protein amounts were used for both tumor and control tissue. Input: sample before affinity purification; Supernatant: unbound material after Neutravidin-purification; NA: Neutravidin.

FIG. 6: Shows two exemplary pairs of non-natural amino acids/labeling reagent of the invention, and their uses for detection of newly synthesized proteins. FIG. 6A shows structures of the AHA-TRA-tag and the HPG-FLA-tag. FIG. 6B shows that dissociated hippocampal neurons (DIV 17) were incubated in either 4 mM AHA, 4 mM methionine (Met), or 4 mM AHA and 40 μM anisomycin (Aniso) for 1 hour, tagged with 1 μM TRA or FLA tag, and immunostained for the dendritic marker protein MAP2. Scale bar=20 μm.

FIG. 7: Shows that two labels, AHA and HPG, can be sequentially applied and detected.

FIG. 8: Shows time-course of protein synthesis in the somata of hippocampal neurons after AHA—(FIG. 8A) or HPG (FIG. 8B) incorporation and [3+2] cycloaddition with fluorescent tags.

FIG. 9: Shows a time-course for the detection of newly synthesized proteins in dendrites of hippocampal neurons.

FIG. 10: Shows a schematic diagram of visualizing endogenous newly synthesized proteins in soma and dendrites of hippocampal neurons.

FIG. 11: Shows immunohistochemical detection of methionyl tRNA synthetase in dendrites.

FIG. 12: Shows local perfusion of dendrites with AHA.

### DETAILED DESCRIPTION OF THE INVENTION

### 1. Overview

Relative to normal cells, rapidly dividing cells (such as those in cancer or other hyper-proliferative cells/tissues) require large amounts of energy and are hot-spots for protein synthesis. Thus labeling the proteome of rapidly proliferating cells would provide a means to differentially mark tumor cells, and distinguish them from metabolically normal cells.

The instant invention provides methods and reagents to use non-natural amino acids for in vitro and/or in vivo labeling, detecting/monitoring/imaging, and/or treatment of hyper-proliferative conditions, such as cancer in an individual. Specifically, bioorthogonal non-canonical amino acids, such as azidohomoalanine (AHA), can cross cell membranes and be used for metabolically labeling of newly synthesized proteins. Therefore, tumors, which show increased protein synthesis, will exhibit a higher incorporation rate of the amino acid (like AHA, which possesses an azide group), and hence harbor significantly more AHA-labeled proteins than other surrounding non-transformed cells. Upon conjugation with a suitable alkyne-tag (which, in the case of AHA can be coupled to the azide group), the tumors can be, among many other things, visualized, made (more) sensitive to irradiation, or destroyed by a drug or pro-drug. In the cases of prodrugs, for example, the alkyne-linked tumor-killing reagent could be “caged” initially, but it will be “uncaged” or enabled to destroy the tumor cells upon secondary activation (infra).

Furthermore, the labeling reagent may bear a moiety for detection and visualization of proteins by means of imaging in the tissue. The moiety for visualization can be used to localize the tumor for irradiation. In another approach, the labeling reagent is either coupled to a “caged” version of a cancer-killing drug, whose release to its reactive “uncaged” form can be locally achieved by secondary treatments. Alternatively, the detection reagent works together with a specialized affinity moiety as an anchor for the drug (which is then administered to the organism).

Thus one aspect of the invention provides a method for detecting and/or treating cancer in a patient, comprising administering to the patient a pharmaceutical composition comprising: (1) a non-natural amino acid comprising a first reactive group; (2) a labeling reagent comprising a second reactive group and a labeling moiety; under conditions where the non-natural amino acid is preferentially incorporated into the newly synthesized proteins of the cancer (including cancer cells and tumor vasculature); wherein the first and second reactive groups react to label the non-natural amino acid with the labeling reagent; wherein the labeling reagent comprises a detectable label, inhibits the progression of the cancer, and/or facilitates the killing of the cancer.

In certain embodiments, the cancer is at least about 10%, 20%, 50%, 100%, 2-fold, 3-fold, 5-fold, 10-fold, 20-fold, 50-fold, 100-fold or more preferentially labeled than a surrounding normal tissue.

As used herein, “differentially or preferentially mark/label,” its equivalents or grammatical variations refers to the fact that certain cells/tissues (such as cancer cells or the endothelial cells of the tumor vasculature) are marked or labeled to a higher degree as compared to others (such as normal cells/tissues). Preferably, the preferentially marked cells or tissues (such as tumor cells or other hyper-proliferating cells) incorporates at least about 10%, 20%, 50%, 100%, 2-fold, 3-fold, 5-fold, 10-fold, 20-fold, 50-fold, 100-fold or more markers/labels than the control/normal cells (e.g., “50% more preferentially marked/labeled,” etc.) when roughly the equivalent amount of cells/tissues are compared. In certain embodiments, the fold of preferential incorporation is measured at a pre-determined or specific time point, such as 5 min., 10-min., 20-min., 30-min., 60 min., 90 min., 2 hrs, 3 hrs, 5 hrs, 12 hrs, 24 hrs, 2 days, 3 days, 1 week, 2 weeks, 1 month or more after exposing or contacting the cells or tissues with the marker or label. In certain embodiments, the fold of preferential incorporation is measured after a steady state of protein synthesis has been reached (e.g., no appreciable further incorporation is observed in a given number of cells after a specific time point).

As used herein, “cancer” or “tumor” are used interchangeably, and includes hyper-proliferative cells and malignant cells capable of invading and/or metastasizing other normal tissues. Cancer also includes endothelial cells or other cells in the tumor vasculature, which may not be malignant per se, but support cancer growth/metastasis.

As used herein, “progression” includes slowing down or reducing the growth, proliferation, invasion, and/or metastasis of cancer. The cancer cells may undergo apoptosis, go into senescence, or simply become necrotic.

According to the methods of the invention, new proteins synthesized in a cell (such as in a cancer cell) are labeled by contacting the cell with a non-natural amino acid that can be incorporated into proteins in the cell in place of a naturally occurring amino acid. The non-natural amino acid generally has a first reactive moiety that can be used to label proteins that incorporate such an amino acid residue. Generally, the first reactive moiety has a functional group that can specifically react with a second reactive moiety on a labeling reagent, which labeling reagent further comprises a functional labeling moiety, such as a fluorescent moiety, an affinity moiety, etc. Once the first reactive moiety on the non-natural amino acid reacts with the second reactive moiety on the reagent, the functional labeling moiety becomes associated with the protein containing the non-natural amino acid, thereby facilitating the labeling, detecting/monitoring, isolating, and/or identifying the translation products in a cell or tissue. The labeling moiety may also be custom-designed to comprise a wide range of cancer-killing agents (infra) to kill cancer cells.

Since the non-natural amino acids can be incorporated into all newly synthesized proteins, including membrane receptors exposed to the extracellular environment, the label moieties need not diffuse inside the target cell to exert their biological effects. Thus in certain embodiments, the labeling reagent may be bulky and non-membrane permeable. This can include certain cancer killing agents that exert their effects from outside the cell.

Preferably, the first and the second reactive moieties react with each other with relatively high specificity, such that the reaction may occur without undesirable side-reactions or interferences due to the presence of other proteins without the non-natural amino acids (e.g., reactions between the reagent and the unlabeled proteins).

The cells may be cultured in vitro, and the non-natural amino acid and the reagent are provided directly to the cell culture medium. The cells may even be lysed or partially permeabilized to facilitate the accessibility of the non-natural amino acid and the reagent to the translation machinery. This embodiment may be useful for large scale or high throughout screening, where test cells are cultured in vitro to assess the effects of a library of test compounds on cell growth or proliferation.

Alternatively, the cells may be cells of a tissue or a live organism (such as a cancer patient), wherein the non-natural amino acid and the reagent are administered to the organism (e.g., by feeding, direct injection, etc.).

In one embodiment, the non-natural amino acid is incorporated into proteins by the endogenous translation machinery of cells or tissue, including the endogenous amino-acyl tRNA synthetases (AARS) that can take the non-natural amino acid as a substrate, and charge it to an endogenous tRNA; and the endogenous ribosome. Preferably, the AARS and/or the tRNA are for a natural amino acid to which the non-natural amino acid is a structural homolog.

Alternatively, the non-natural amino acid is incorporated into proteins of the cells/tissue with at least partial aid from non-endogenous translation machinery. For example, the non-natural amino acid may be charged to a tRNA (endogenous, modified endogenous, or tRNA from a different species, etc.) by a non-endogenous AARS, which AARS may be engineered specifically to take the non-natural amino acid as a substrate. See, for example, US 2004-0053390 A1, US 2004-0058415 A1, WO 03/073238 A2, U.S. Pat. No. 6,586,207 (all incorporated herein by reference). The charged non-natural amino acid—tRNA may be produced in vitro and provided to the cell or tissue. Alternatively, the modified AARS and/or tRNA may be provided to the cells/tissue, for example, by introducing into the cells a polynucleotide encoding the AARS and/or tRNA, to charge the non-natural amino acid in vivo. These may be useful for in vitro large scale screening methods of the invention.

The non-natural amino acid preferably contains one or more of the following desirable characteristics: (1) relatively permeable through bio-membranes, such as plasma membranes, such that it can be directly provided in tissue culture medium or administered to a live organism for direct uptake by the cell; (2) relatively stable in vitro and in vivo; (3) being a structural homolog of one or more natural amino acids, such as methionine or phenylalanine (In certain embodiments, the natural amino acid is an essential amino acid, so that the cells can be made auxotrophic for that natural amino acid; in certain embodiments, the natural amino acid is relatively rarely used in proteins); (4) capable of being charged directly to an endogenous tRNA by an endogenous AARS; (5) the charged non-natural amino acid—tRNA complex can be readily incorporated into proteins by endogenous ribosomes; (6) the structure of the non-natural amino acid is such that, upon incorporation into the proteins, it does not substantially affect the folding and/or biological function of any proteins incorporating the non-natural amino acid; and (7) being substantially non-toxic and biologically inert.

In certain embodiments, the first reactive moiety on the non-natural amino acid preferably can react with the second reactive moiety of another reagent under relatively mild conditions, such as physiological conditions with a relatively neutral/physiological pH and temperature. One or more catalysts (preferably non-toxic) may also be provided to facilitate the reaction under such conditions.

However, in certain other embodiments, the reaction may occur in more harsh conditions, with any type of suitable catalyst, since proteins labeled by the non-natural amino acid may be isolated or be present in a cell lysate or an in vitro translation system before the reaction between the first and second reactive moieties occur.

In certain embodiments, cells/tissues incorporated with non-natural amino acids may be fixed or permeated after the incorporation step, before the agent with the second reactive group and functional moieties are provided (e.g., in the case of immunostaining, etc.). Since the reaction between the first and the second reactive groups occur only after the fixation of cells/tissues, there are fewer limitations regarding the type of second reactive groups and/or catalysts that may be used in the methods of the invention.

In certain embodiments, the methods of the invention can be used to incorporate non-natural amino acids (e.g., those with azide moiety, such as AHA, or those with terminal alkyne groups) into proteins/polypeptides translated in vitro. In these embodiments of the invention, either endogenous or ortholog AARS/tRNA pairs may be used to charge the non-natural amino acids. Since there is no issue of membrane permeability, and much less of an issue of toxicity (if any at all), the range of non-natural amino acids and the types of catalysts that can be used in these embodiments are wider than those for intact cell/tissue use.

In one embodiment, the first reactive group is an azido group. For example, the amino acid used may be azidohomoalanine (AHA) or an analog thereof, such as azidoalanine, azidonorvaline, or azidonorleucine (see below). AHA is an analog of the essential natural amino acid methionine, which is relatively rarely used in proteins. Other methionine/AHA homologs that can be used in the invention include: azidoalanine, azidonorvaline, and azidonorleucine, etc. See Link et al., J. Am. Chem. Soc. 126: 10598-10602, 2004 (incorporated herein by reference).

AHA was not toxic to cells, including the less robust and more fragile mammalian cell type, primary cultured postnatal neurons, as indicated by healthy neuronal processes and the absence of abnormal varicosities in the dendrites (see Dieterich et al., Proc. Natl. Acad. Sci. U.S.A. 103(25): 9482-87, 2006). It can be incorporated into a wide variety of cellular proteins, and there is no evidence that AHA exposure alters global protein synthesis rates (Dieterich et al., supra). In addition, AHA incorporation does not cause severe protein misfolding or degradation, and there is no increase in the ubiquitination of total protein in AHA-treated cells when compared to buffer or methionine controls (Dieterich et al., supra). Experiments conducted in vivo have also demonstrated that azides that can be used for metabolic labeling are non-toxic to live animals (see Dube et al., Proc. Natl. Acad. Sci. U.S.A. 103(13): 4819-4824, 2006).

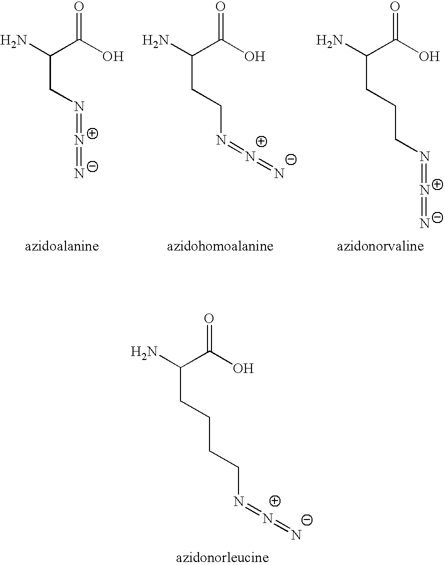

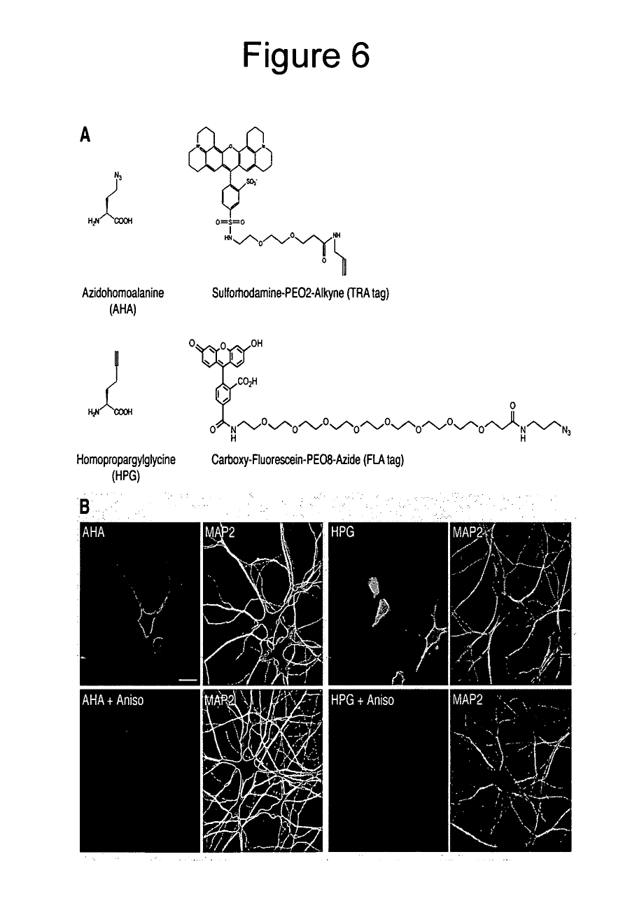

In another embodiment, the non-natural amino acid has a ketone moiety. In yet another embodiment, the non-natural amino acid has a diboronic acid moiety. In yet another embodiment, the non-natural amino acid has a terminal alkyne moiety (see, for example, Homopropargylglycine or HPG in FIG. 6).

In certain embodiments, the non-natural amino acid is AHA or HPG, while the labeling reagent is sulforhodamine-PEO2-Alkyne (TRA tag) or carboxy-Fluorescein-PEO8-Azide (FLA tag), respectively (see FIG. 6).

In certain embodiments, the non-natural amino acid is site-specifically incorporated in place of a natural amino acid selected from methionine or phenylalanine.

The concentration of the non-natural amino acid used to contact cells in culture (or the amount administered to an animal) can be selected to optimize the detection of newly synthesized proteins.

Once the non-natural amino acid is incorporated into the proteins of the cells, proteins from the cells are then treated with a reagent that can become associated with the non-natural amino acid. The reagent comprises a second reactive moiety that can react with the first reactive moiety of the non-natural amino acid. For example, when the non-natural amino acid has an azido moiety, the reagent will generally have an alkyne moiety, and vice versa.

In certain embodiments, the reagent is an affinity reagent that also comprises at least one affinity moiety, such as biotin, that can be used to isolate the non-natural amino acid-labeled proteins. In one embodiment, the affinity reagent further comprises a second affinity moiety, such as an antigenic moiety that can be recognized by an antibody. In a preferred embodiment, the affinity reagent has a FLAG epitope that can also be used for affinity purification. In a preferred embodiment, the affinity reagent has both a FLAG tag and a biotin moiety. In such embodiments, the affinity moieties may be used independently or in combination (e.g., sequential) to effect optimum isolation of the labeled protein through the affinity moieties.

As used herein, “affinity moiety” includes any moiety that may be used for affinity binding/isolation/purification purpose. Common affinity moieties include antigens (e.g., epitope tags, such as FLAG tag, HA tag, His6 tag, etc.) for certain antibodies, a member of a ligand-receptor pair, etc. The affinity moiety may be polypeptide, nucleic acid, polysaccharide, lipids, vitamin (e.g., biotin, etc.), or molecules of any other chemical nature.

In certain embodiments, the reagent is a fluorescent reagent that comprises the second reactive moiety and one or more fluorescent moieties. Preferably, the fluorescent moiety becomes more fluorescent when the first and the second reactive moieties react with each other, or the unreacted fluorescent reagent can be easily removed (e.g., washed away).

In certain embodiments, the labeling reagent is a cancer killing reagent. There are many cancer killing reagents that may be used in custom designed labeling reagents. The instant specification described some of the representative agents that are for illustrative purpose only.

For example, the labeling reagent may comprise a chelate moiety for chelating a metal. Thus the labeling reagent may be a chelator for a radiometal or a paramagnetic ion. In certain embodiments, an effective amount of chelator compounds may be infused into the patient. Suitable chelator compounds include EDTA or DTPA.

In certain embodiments, the labeling reagent may be a chelator for a radionuclide useful for radiotherapy or imaging procedures. The radionuclide may be a beta- or alpha-emitter for radio-therapeutic use. Or the radionuclide may be a gamma-emitter, positron-emitter, Auger electron-emitter, X-ray emitter or fluorescence-emitter.

A preferred radionuclide is ^{99m}Tc (technium).

In certain embodiments, the labeling reagent comprises a bifunctional chelator NₓS_{y} that are capable of coordinately binding a metal or radiometal, wherein x and y are integers between 1 and 4. For example, NₓS_{y} may have a N₂S₂ or a N₃S core.

In certain embodiments, the labeling reagent comprises a cytotoxic moiety. The cytotoxic moiety may be a radiosensitizing agent, a Boron addend, a chemotherapeutic agent, a protein synthesis inhibitor, a prodrug activated by host metabolism, a cytotoxic toxin, an enzyme that converts prodrug locally, or a dye used in photodynamic therapy or in conjunction with appropriate non-ionizing radiation.

For example, the radiosensitizing agent may be selected from: nitroimidazoles, metronidazole or misonidazole.

The Boron addend may be carborane.

The chemotherapeutic agent may include: taxol; nitrogen mustards; ethylenimine derivatives; alkyl sulfonates; nitrosoureas; triazenes; pyrimidine analogs; purine analogs; vinca alkaloids; antibiotics; enzymes; platinum coordination complexes; substituted urea; methyl hydrazine derivatives; adrenocortical suppressants; or hormones and antagonists selected from: adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), or androgens (testosterone propionate and fluoxymesterone).

The protein synthesis inhibitor may be puromycin, cycloheximide, or ribonuclease.

The labeling reagent may also comprise a prodrug that is only activated from its inactive precursor form by host metabolism. For example, the enzyme that converts prodrug locally is alkaline phosphatase, and the prodrug is etoposidephosphate.

The cytotoxic toxin may be selected from: ricin, ricin A chain (ricin toxin), Pseudomonas exotoxin (PE), diphtheria toxin (DT), Clostridium perfringens phospholipase C (PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin or volkensin.

Since the cytotoxic moiety is selectively delivered to the target cell, it may be administered to the patient at a dose that contain 10-100 times less active agent as an active moiety than the dosage of agent administered as unconjugated active agents.

In certain embodiments, the reagents may be cleavable. In one embodiment, the reagent harbors, in addition to any of the above-described components (such as an antigenic component), a cleavage site for a sequence-specific protease, such as Factor Xa or PreScission Protease. When used as an affinity tag, a first purification can be performed that will select for the presence of the affinity reagent. For example, the proteins can be purified using a FLAG binding resin. Upon treatment with the specific protease, only proteins that have the non-natural amino acid will be released for subsequent analyses, while proteins that bind non-specifically to the affinity reagent or the resin/column will likely stay bound.

In another embodiment, the reagent may comprise a photo-cleavable linker between the second reactive moiety and the functional group. For example, a photo-cleavable affinity reagent includes a photo-labile linker between the second reactive moiety and the one or more affinity moieties. Upon light exposure, non-natural amino acid-bearing molecules are specifically released and can be subsequently identified in various detection assays, such as mass spectrometric analyses.

Proteins or fragments thereof having the non-natural amino acid moiety can be isolated using the one or more affinity moieties, if such moieties are present in the reagent comprising the second reactive moiety. The isolated proteins can be analyzed further. For example, such proteins or fragments can be identified by mass spectrometry techniques, with or without obtaining the sequences of the proteins/fragments. In certain embodiments, the isolated proteins can be digested using a protease, such as trypsin, and the resulting peptides can be analyzed by mass spectrometry. Alternatively, proteins from the cell may be digested with protease before the resulting fragments are reacted with the second reactive groups. The resulting peptide fragment—affinity reagent complex can then be isolated through affinity column. Protein digestion may also be carried out after the reaction with the second reactive moiety, but before the affinity purification/isolation step.

In certain embodiments, the cells may be treated with a second non-natural amino acid, and/or a natural amino acid derivative. In one embodiment, the natural amino acid derivative contains one or more isotopes such that the overall molecular weight of the amino acid derivative is different from that of a wild-type natural amino acid. For example, the natural amino acid derivative may be a deuterated amino acid (alternatively, an amino acid that contains ³H), which gives a mass shift in mass spectrum. In certain embodiments, the non-natural amino acid may also contain isotopes, such as ¹³C, ¹⁵N or ¹⁸O. The presence of such isotopes may help to differentiate newly synthesized proteins from different samples, thus enabling simultaneous analysis of multiple samples in, for example, high throughput experiments.

In certain embodiments, the patients may be administered a second pharmaceutical composition comprising: (3) a second non-natural amino acid comprising a third reactive group, under conditions where the second non-natural amino acid is preferentially incorporated into the newly synthesized proteins of the cancer; (4) a second labeling reagent comprising a fourth reactive group and a second labeling moiety, wherein the third and fourth reactive groups react to label the second non-natural amino acid with the second labeling reagent; wherein at least one of the labeling reagents inhibits the progression and/or facilitates the killing of the cancer.

For example, new protein synthesis in the cells may be labeled first with AHA and a first labeling moiety, then the same cells are labeled with HPG and a second labeling moiety. The two moieties may be two different cancer killing drugs that, when used together, exhibit synergistic cancer killing. Alternatively, one labeling moiety may be a prodrug, while the other labeling moiety may be an enzyme that converts the prodrug to an effective cancer killing drug. Only cells having both types of labels are preferentially killed by the therapy. In yet another embodiment, one of the labeling group may be a cancer killing drug, while the other is an imaging reagent (e.g., radioactive or fluorescent markers, etc.) that will facilitate the monitoring of cancer killing effect over the course of treatment.

The methods of the invention can be used in general to profile the expression pattern of new proteins in two or more samples/cell lines/tissues, and compare such expression patterns.

In one embodiment, the methods of the invention may be used to study pharmaceutical impact on protein expression in a cell. According to such a method, the protein expression pattern is determined according to the methods of the invention in the presence or absence of a pharmaceutical agent or a candidate drug.

Thus another aspect of the invention provides a high throughput screening method for identifying a compound that inhibits cell proliferation, the method comprising: (1) contacting a control cell with a non-natural amino acid comprising a first reactive group, under conditions where the non-natural amino acid is preferentially incorporated into the newly synthesized proteins of the control cell; (2) contacting a control cell with a labeling reagent comprising a second reactive group and a labeling moiety, wherein the first and second reactive groups react to label the non-natural amino acid with the labeling reagent; (3) repeat steps (1) and (2) to label a test cell at the presence of a candidate compound; (4) comparing the quantity of the labeling moiety in the control cell and the test cell, respectively; wherein a decrease in quantity of the labeling moiety in the test cell is indication that the candidate compound inhibits proliferation of the test cell.

In certain embodiments, the control cell and the test cell are primary cancer cells from the same cancer, or are from the same cancer cell line.

To ensure that the candidate compound is not just generally toxic to all cells, the method may further comprise testing the general toxicity of the candidate compound on a normal or healthy cell. Preferred candidates would exhibit cancer-specific killing or inhibition of proliferation without substantially damaging normal tissues.

In certain embodiments, the labeling moiety is a fluorescent moiety or a reagent that can be subsequently coupled to a fluorescent reagent, and wherein step (4) is effectuated by monitoring fluorescent intensity.

In one embodiment, the methods of the invention may be used to monitor the changes in expression pattern, if any, of a sample over time. For example, a zero time-point expression pattern may be obtained before the sample is subject to certain treatment. Expression patterns at later time points may be obtained and compared to the zero time point. Pulse labeling of new proteins using the method of the invention may be used to obtain samples from different time points.

The methods of the invention can also be used to metabolically label new protein synthesis in cells or tissues. Such method is particularly useful for real-time imaging of local protein synthesis in cells/tissues. For example, in one embodiment, protein synthesis can be monitored within a living cell, such as one in a live organism or in tissue culture. According to such methods, the cell is contacted with a non-natural amino acid, such as AHA, that is incorporated in a protein in place of a naturally occurring amino acid by the cell's endogenous machinery. The cells are then treated with a fluorescent reagent that has a second reactive moiety capable of reacting with the first reactive moiety on the non-natural amino acid residue to form a covalent bond or, alternatively, the cells are treated with a reagent that allows for sequential coupling of a fluorescent moiety. The reaction of the fluorescent tags with the proteins will result in fluorescent-labeled proteins. In a preferred embodiment, the fluorescent reagent is substantially more fluorescent after reaction with the non-natural amino acid residue.

The invention also includes various reagents for use with the methods of the invention. In certain embodiment, the invention provides any of the cancer killing labeling reagents of the invention (see above and more details below). Preferably, such cancer killing labeling reagents are formulated as pharmaceutical compositions for use in human or non-human animals.

In certain other embodiments, the invention provides labeling reagents that comprise a first reactive moiety capable of reacting with a non-natural amino acid (such as one that has been incorporated into a protein), at least one labeling group/moiety, and optionally at least one cleavage site that allows the separation of the second reactive moiety and the labeling moiety. The labeling moiety may be any of the cancer killing moieties described herein or known in the art.

In other embodiments, the labeling moiety is an affinity moiety, and the affinity reagent has two affinity groups/moieties and two distinct cleavage sites. In one such embodiment, the affinity reagent has two affinity moieties, including a biotin group and an immunological/epitope tag, and a two peptide cleavage sites (such as two protease cleavage sites for trypsin or trypsin-like proteases).

As used herein, the term “physiological conditions” is meant to encompass those conditions compatible with living cells, e.g., predominantly aqueous conditions of a temperature, pH, salinity, etc. that are compatible with living cells.

The term “aryl” as used herein means 5- and 6-membered single-aromatic radicals which may include from zero to four heteroatoms. Representative aryls include phenyl, thienyl, furanyl, pyridinyl, (is)oxazoyl and the like.

The term “lower alkyl”, alone or in combination, generally means an acyclic alkyl radical containing from 1 to about 10, preferably from 1 to about 8 carbon atoms and more preferably 1 to about 6 carbon atoms. Examples of such radicals include methyl, ethyl, n-propyl, isopropyl, n-butyl, isobutyl, sec-butyl, tert-butyl, pentyl, iso-amyl, hexyl, octyl and the like.

Another aspect of the invention provides a kit, comprising: (1) one or more non-natural amino acids capable of being incorporated into newly synthesized proteins in a translation system, such as in vivo in an animal, in vitro in tissue culture systems, or in a cell-free in vitro translation system (optionally supplemented with modified tRNA and/or tRNA AARS); and (2) one or more labeling reagents of the invention, such as a subject cancer killing labeling reagent comprising a cancer killing moiety (supra).

In a preferred embodiment, the kit comprises two or more pairs of non-natural amino acids/labeling reagents, where the labeling moieties on the two label reagents are different (for example, different fluorescent markers, drugs or other cancer killing agents, etc.)

The kit of the invention may be provided as a therapeutic kit for treating a patient (e.g., a cancer patient) using the methods of the invention.

The kit of the invention may be provided as a diagnostic kit for detecting cancer or other fast proliferating cells in a patient (e.g., a cancer patient) using the methods of the invention.

The kit of the invention may also be provided as a research kit for in vitro cell labeling using the methods of the invention. In a preferred embodiment, the cell is a neuronal cell, such as cultured primary neurons, neuronal cell lines, or stem/progenitor cells committed to the neuronal differentiation pathway.

The kits of the invention may further comprise an instruction or label for the intended use of the kit.

Another aspect of the invention provides a method for detecting or imaging cancer in a patient, comprising: (a) contacting the patient with a non-natural amino acid comprising a first reactive group, under conditions where the non-natural amino acid is preferentially incorporated into the newly synthesized proteins of the cancer; (b) contacting the patient with a labeling reagent comprising a second reactive group and a labeling moiety, wherein the first and second reactive groups react to label the non-natural amino acid with the labeling reagent; (c) detecting the labeling moiety, thereby detecting cancer in the patient.

In certain embodiments, the labeling moiety comprises an imaging agent.

In certain embodiments, the imaging agent is a radionuclide imaging agent.

In certain embodiments, the radionuclide imaging agent is radioactive iodine or indium.

In certain embodiments, the labeling moiety is detected by radioscintigraphy, magnetic resonance imaging (MRI), computed tomography (CT scan), or positron emission tomography (PET).

In certain embodiments, the contacting step (a) or (b) is independently effected by administering to the patient the non-natural amino acid or the labeling reagent orally, perentarally, transdermally, topically, intramuscularly (i.m.), intraperitoneally (i.p.), or intraveneously (i.v.).

In certain embodiments, the detecting step (c) includes determining the volume, shape and/or location of the cells labeled by the labeling reagent in the patient.

The sections below provides more details of certain features of the invention. All embodiments described are generally considered to be able to combine with any other embodiments where applicable.

### 2. Non-natural Amino Acids

Numerous non-natural amino acids have been successfully incorporated into protein either in vitro (such as in vitro translation) or in vivo (e.g., in live cells or animals). These non-natural amino acids usually contain one or more functional groups not present in the twenty naturally occurring amino acids, thus incorporation of non-natural amino acids into proteins in vivo can provide biological materials with new chemical functions and improved physical properties. Examples include new posttranslational modification chemistry by introducing azide and ketone moieties into recombinant proteins, and novel strategies for engineering hyper-stable proteins by incorporating fluorinated side chains.

However, implementing such methods generally requires certain manipulation of the protein biosynthesis machinery. This may be accomplished by using a cell's translational machinery in either a residue-specific (Link et al., Curr. Opin. Biotechnol. 14: 603-9, 2003) or a site-specific manner (Wang & Schultz, Angew. Chem. Int. Edn. Engl. 44: 34-66, 2004).

Thus in one embodiment, residue-specific incorporation of non-natural amino acids into proteins involves replacement of a natural residue with a conservatively modified analog. The translational machinery is sufficiently tolerant of altered substrates that, especially in the absence of competing natural substrates, the modified residue is converted to an aminoacyl tRNA that is subsequently used by the ribosome. Alternatively, the specificity of certain endogenous or designed/engineered exogenous AARS may be altered to catalyze the attachment of the non-natural amino acids to suitable tRNAs.

By these mechanisms, non-natural amino acids bearing bioorthogonal chemical moieties can be introduced into proteins that are overexpressed in a host cell. To avoid competition with the endogenous amino acid, the host cell (e.g., bacterial strain) may be rendered auxotrophic for the natural amino acid. Proteins cannot be overexpressed unless the cells are supplemented with either that residue or a closely related unnatural analog. For example, a phenylalanine auxotroph was used to express proteins in which all phenylalanine residues were replaced with p-azidophenylalanine or p-acetylphenylalanine (a keto derivative) (Datta et al., J. Am. Chem. Soc. 124: 5652-5653, 2002; Kirshenbaum, ChemBioChem 3: 235-237, 2002). Similarly, a methionine auxotroph was used for production of proteins that contained homopropargylglycine or azidohomoalanine at sites that encode for methionine (van Hest, J. Am. Chem. Soc. 122: 1282-1288, 2000; Kiick et al., Proc. Natl. Acad. Sci. USA 99: 19-24, 2002). Applicants have also extended this work to the labeling of bacterial cell surfaces (Link, J. Am. Chem. Soc. 126: 10598-10602, 2004; Link & Tirrell, J. Am. Chem. Soc. 125: 11164-11165, 2003). Azido amino acids were installed in outer membrane protein C (OmpC) of an E. coli methionine auxotroph and the cell surface azides were then ligated with alkyne probes through both copper(I)-mediated and strain-promoted [3+2] cycloaddition.

Residue-specific metabolic labeling can produce proteins with multiple copies of a bioorthogonal functional group, and is particularly useful for the proteome-wise expression profiling. However, this has only limited application in cases where a chemical moiety is desired at a single position within the protein. Thus site-specific insertion of a bioorthogonal amino acid may be achieved using nonsense suppression techniques (Wang & Schultz, Angew. Chem. Int. Edn. Engl. 44: 34-66, 2004). In this approach, a mutually selective tRNA and aminoacyl-tRNA synthetase are developed so that the non-natural amino acid can be uniquely activated by the tRNA in vivo. The tRNA's anticodon is engineered to complement a rare stop codon, which is co-opted to encode the non-natural amino acid in the corresponding DNA (and intermediate mRNA). Cells transfected with genes encoding the engineered tRNA, aminoacyl-tRNA synthetase and target protein will produce the modified protein when supplemented with the non-natural amino acid.

The non-natural amino acid mutagenesis method has been used to introduce chemical moieties into proteins in both E. coli and yeast. For example, m-acetylphenylalanine was site-specifically incorporated into LamB, an outer-membrane protein of E. coli, and subsequently labeled with membrane-impermeant hydrazide dyes (Zhang et al., Biochemistry 42: 6735-6746, 2003). Similarly, azido and alkynyl amino acids related to tyrosine were installed in proteins within both E. coli and yeast. After cell lysis, the derivatized proteins were tagged by copper-catalyzed [3+2] cycloaddition.

Regardless of how the non-natural amino acid is incorporated into proteins, in certain embodiments, it is preferred that the non-natural amino acids contain one or more of the following desirable characteristics to facilitate easy incorporation into live cells in vitro or in vivo: (1) relatively permeable through bio-membranes, such as plasma membranes, such that it can be directly provided in tissue culture medium or administered to a live organism for direct uptake by the cell; (2) relatively stable in vitro and in vivo; (3) being a structural homolog of one or more natural amino acids, such as methionine or phenylalanine.

In certain embodiments, the natural amino acid is an essential amino acid, so that the cells are auxotrophic for that natural amino acid; in certain embodiments, the natural amino acid is relatively rarely used in proteins); (4) capable of being charged directly to an endogenous tRNA by an endogenous AARS; (5) the charged non-natural amino acid—tRNA complex can be readily incorporated into proteins by endogenous ribosomes; (6) the structure of the non-natural amino acid is such that, upon incorporation into the proteins, it does not substantially affect the folding and/or biological function of any proteins incorporating the non-natural amino acid; and (7) being substantially non-toxic and biologically inert. The non-natural amino acid is typically relatively small in size, and contains a single functional reactive moiety.

In one preferred embodiment of the invention, the non-natural amino acid is incorporated in the place of a naturally occurring amino acid. In general, such non-natural amino acid will be an acceptable substrate for an aminoacyl-tRNA synthetase (AARS) that charges a tRNA recognizing a naturally occurring codon. Preferably, the AARS and/or the tRNA are endogenous AARS and endogenous tRNA.

For in vivo use, or in vitro use in live cells, the non-natural amino acid chosen should not be significantly toxic to cells.

However, in certain other embodiments, the methods of the invention also can be utilized in cells, tissues or organisms engineered to express an aminoacyl-tRNA that charges a natural tRNA with the non-natural amino acid. The methods of the invention may further be used in in vitro translation systems where cell lysates are used to support protein synthesis. In those embodiments, the requirement of membrane permeability becomes largely irrelevant, and one or more ortholog AARS/tRNA pairs may be provided to the lysate to support incorporation of the non-natural amino acid.

There are several preferred reactive moiety pairs that are particularly suitable for the invention, which are illustrated below as non-limiting examples.

(A) Azide—Alkyne Ligation

In one specific embodiment, the protein tagging is based on the azide-alkyne ligation. In a typical embodiment, the side chain of the non-natural amino acid contains the azide group, while the second reactive moiety on the reagent contains the terminal alkyne moiety. Alternatively, the side-chain of the non-natural amino acid contains the terminal alkyne, while the second reactive moiety contains the azide moiety.

Several non-natural amino acids contain the azide group and are suitable for the instant invention. For example, azidoalanine, azidohomoalanine (AHA), azidonorvaline, or azidonorleucine are all azide-containing methionine homologs that can be incorporated into proteins using the endogenous protein translation machinery. AHA, which serves as a surrogate for the essential amino acid methionine during protein synthesis, exhibits excellent membrane permeability and is not toxic even in primary neuronal cell cultures. Studies in E. coli and experiments in mammalian cells have shown no evidence of increased protein degradation upon introduction of AHA, indicating that the modified amino acid is not likely to cause severe protein misfolding.

Azides are suitable for labeling all classes of biomolecules (including proteins) in any biological locale. This versatile functional group is absent from nearly all naturally occurring species. (Only one naturally occurring azido metabolite has been reported to date, isolated from unialgal cultures. See Griffin, Prog. Med. Chem. 31: 121-232, 1994). Azides do not react appreciably with water and are resistant to oxidation. Additionally, azides are mild electrophiles that do not react with amines or the other “hard” nucleophiles that are abundant in biological systems. Rather, they require “soft” nucleophiles for reaction. Although azides are susceptible to reduction by free thiols, including the ubiquitous cellular reductant, glutathione, reactions between monothiols and alkyl azides typically require vigorous heating (100° C. for several hours) or auxiliary catalysts.

Azides are prone to decomposition at elevated temperatures, but they are quite stable at physiological temperatures (See Griffin, Prog. Med. Chem. 31: 121-232, 1994). Whereas aryl azides are well-known photocrosslinkers, alkyl azides do not photodecompose in the presence of ambient light. Finally, although azide anion (for example, in the form of NaN₃) is a widely used cytotoxin, organic azides have no intrinsic toxicity. Indeed, organic azides are components of clinically approved drugs such as AZT (See Griffin, Prog. Med. Chem. 31: 121-232, 1994).

Although kinetically stable, azides are predisposed to unique modes of reactivity owing to their large intrinsic energy content. This feature has been exploited for the development of bioorthogonal reactions, including the Staudinger ligation of azides with functionalized phosphines and the [3+2] cycloaddition of azides with activated alkynes. These reactions can be used for the selective labeling of azide-functionalized biomolecules.

Staudinger ligation. In 1919, Hermann Staudinger reported that azides react with triphenylphosphines (soft nucleophiles) under mild conditions to produce aza-ylide intermediates (Staudinger & Meyer, Helv. Chim. Acta 2: 635-646, 1919). Bertozzi et al. modified the classic Staudinger reaction by introduction of an intramolecular trap into the phosphine (Saxon & Bertozzi, Science 287: 2007-2010, 2000). Now known as the Staudinger ligation, this transformation ultimately produces a covalent link between one nitrogen atom of the azide and the triarylphosphine scaffold (see below).

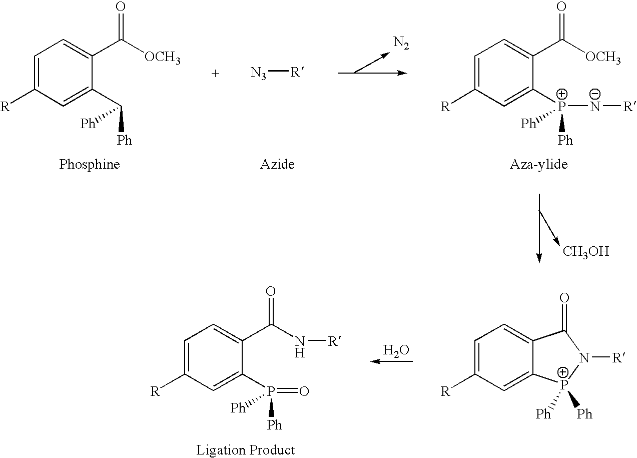

### The Staudinger Ligation

The Staudinger ligation can be used to covalently attach probes to azide-bearing biomolecules. Like the azide, phosphines do not react appreciably with biological functional groups and are therefore also bioorthogonal. Additionally, the reaction proceeds readily at pH 7 with no apparent toxic effects. These and other applications of the Staudinger ligation have been recently reviewed (see Kohn & Breinbauer, The Staudinger ligation—a gift to chemical biology. Angew. Chem. Int. Edn. Engl. 43: 3106-3116, 2004).

Copper-catalyzed [3+2] azide-alkyne cycloaddition. In the context of the Staudinger ligation, the azide serves as an electrophile subject to reaction with soft nucleophiles. Azides are also 1,3-dipoles that can undergo reactions with dipolarophiles such as activated alkynes. These π-systems are both extremely rare and inert in biological systems, further enhancing the bioorthogonality of the azide along this reaction trajectory.

The [3+2] cycloaddition between azides and terminal alkynes to provide stable triazole adducts was first described by Huisgen more than four decades ago (Huisgen, Angew. Chem. Int. Edn. Engl. 2: 565-598, 1963). The reaction is thermodynamically favorable by 30-35 kcal/mol. The reaction typically requires alkyne activation, which process requires elevated temperatures or pressures that are not compatible with living systems. Although this may not be a concern for in vitro use, activation may be achieved with catalysts for use in vitro as well as in vivo.

In one embodiment, the activation may be achieved by the addition of electron-withdrawing groups, such as esters, to the alkyne.

In another embodiment, a Cu(I)-based catalyst may be used to accelerate the rate of cycloaddition between azides and alkynes by about 10⁶-fold (Rostovtsev et al., Angew. Chem. Int. Edn. Engl. 41: 2596-2599, 2002; Tornoe, J. Org. Chem. 67: 3057-3064, 2002). This copper-catalyzed reaction, termed “click” chemistry, proceeds readily at physiological temperatures and in the presence of biological materials to provide 1,4-disubstituted triazoles with nearly complete regioselectivity (Kolb & Sharpless, Drug Discov. Today 8: 1128-1137, 2003). The copper-mediated reaction has been used to tag azides installed within virus particles (Wang et al., J. Am. Chem. Soc. 125: 3192-3193, 2003), nucleic acids (Seo et al., Proc. Natl. Acad. Sci. USA 101: 5488-5493, 2004) and proteins from complex tissue lysates (Speers & Cravatt, ChemBioChem 5: 41-47, 2004) with virtually no background labeling.

It should be noted that the same reaction can be carried out in reverse—using the alkyne as the first reactive moiety on the non-natural amino acid. Like the azide, a terminal alkyne consists of a mere three atoms.

The primary advantage of the catalyzed azide-alkyne cycloaddition over the Staudinger ligation is its faster rate. The copper-catalyzed reaction of azides with alkynes reportedly proceeds at least 25 times faster than the reaction of azides with triarylphosphines in cell lysates. Accordingly, “click” chemistry is preferably used in situations that require detection of very small quantities of azide-labeled biomolecules.

In certain embodiments, an improved protocol for copper-catalyzed triazole formation may be used to further increase the efficiency of reaction by about 10-fold (Link et al., J. Am. Chem. Soc. 126: 10598-10602, 2004, incorporated herein by reference).

According to this embodiment of the invention, instead of generating Cu(I) in situ by combining Cu(II) and a reducing agent (such as 200 μM CuSO₄ and 200 μM of tris-(carboxyethyl)phosphine (TCEP)), ultra-pure Cu(I) is added directly to the reaction mixture as catalyst. In a preferred embodiment, the ultra-pure Cu(I) is CuBr, with at least 99.999% purity (such as those obtained from Aldrich).

Strain-promoted cycloaddition. An alternative means of activating alkynes for catalyst-free [3+2] cycloaddition with azides involves the use of ring strain (Agard et al., J. Am. Chem. Soc. 126: 15046-15047, 2004). Constraining the alkyne within an eight-membered ring creates ˜18 kcal/mol of strain, much of which is released in the transition state upon [3+2] cycloaddition with an azide (Turner et al., J. Am. Chem. Soc. 95: 790-792, 1972). As a consequence, cyclooctynes react with azides at room temperature, without the need for a catalyst. This strain-promoted cycloaddition has been used to label biomolecules both in vitro and on cell surfaces without observable toxic effects (Agard et al., J. Am. Chem. Soc. 126: 15046-15047, 2004). The rate of the strain-promoted cycloaddition can be increased by appending electron-withdrawing groups to the octyne ring.

(B) Ketones and Aldehydes

A second group of non-natural amino acids that can be incorporated into proteins according to the method of the instant invention includes those with a ketone or aldehyde side chain. Comprising only a handful of atoms, ketones and aldehydes are bioorthogonal chemical moieties that can tag not only proteins, but also glycols and other secondary metabolites. These mild electrophiles are attractive choices for modifying biomolecules as they are readily introduced into diverse scaffolds, absent from endogenous biopolymers and essentially inert to the reactive moieties normally found in proteins, lipids and other macromolecules. Although these carbonyl compounds can form reversible Stiff bases with primary amines such as lysine side chains, the equilibrium in water favors the carbonyl. By contrast, the stabilized Stiff bases with hydrazide and amino groups (hydrazones and oxides, respectively) are favored in water and are quite stable under physiological conditions (Jencks, J. Am. Chem. Soc. 81: 475-481, 1959).

In one embodiment, ketone (and aldehyde) condensations are used for chemical modifications in the presence of cultured cells.

In one embodiment, ketone (and aldehyde) condensations are used to label proteins on cell surfaces or in the extra cellular environment.

In other embodiments, ketone (and aldehyde) condensations are used in the context of selectively labeling tissues in living organisms.

### 3. Labeling Reagents with Second Reactive Moiety

The reagents with the second reactive moiety of the invention comprise at least two moieties: a second reactive group that can specifically react with the first reactive moiety of the non-natural amino acid, and a functional moiety that becomes attached to the incorporated non-natural amino acid after the reaction. Optionally, a cleavable functional group is situated between the second reactive moiety and the functional moiety to allow separation of these two moieties under controlled conditions (e.g., protease digestion, photo-cleavage, etc.).

A number of possible functional moieties can be linked to the second reactive moiety. For example, the functional moiety may be an affinity moiety that can be used to isolate the labeled proteins or fragments thereof.

Alternatively, any other functional groups, such as a radioactive moiety (e.g., radioisotope), a fluorescent moiety, etc., may also be attached to the labeled protein or fragments thereof containing the non-natural amino acids.

(A) Second Reactive Moiety

In one embodiment, when the non-natural amino acid used is AHA or another azide-bearing non-natural amino acid, proteins bearing one or more azide groups can subsequently be tagged for enrichment by treatment with an alkyne-bearing affinity reagent. Alternatively, as described above, the affinity reagent can have a phosphine group for reacting with the azide moiety via a Staudinger reaction (Saxon and Bertozzi, Cell Surface Engineering by a Modified Staudinger Reaction, Science 287: 2007-2010, 2000, incorporated herein by reference).

As used herein, “azide” group refers to R—N₃, wherein R represents the rest of the azide-containing molecule (comprising the non-natural amino acid side chain).

“Terminal alkyne” refers to R′—C≡C, wherein the [3+2] cycloaddition product of the R—N₃+R′—C≡C reaction has a general formula of:

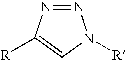

When “strain-promoted cycloaddition is used, the terminal alkyne in the above reaction is replaced by the following reactive partner:

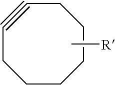

In this case, the final ligation product of the cycloaddition is:

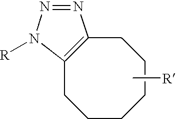

In other embodiments, where the non-natural amino-acid has a ketone group, the affinity reagent will preferably have an aminooxy or hydrazide moiety (NH₂—NH—CO—R′) (See, for example, U.S. Pat. Appln. No. 20020016003 to Bertozzi).

As used herein, “ketone” group refers to the general formula: R—CO—R₂; “aldehyde” group refers to the general formula R—CHO.

“Aminooxy” moiety refers to NH₂—O—R′. The reaction product between the aminooxy group and the ketone/aldehyde group is:

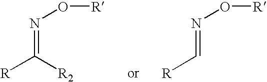

(B) Functional Moiety/Labeling Moiety

Numerous labeling moieties may be covalently linked to or non-covalently associated with the second reactive group of the subject labeling reagents. This and the following sections provide certain (non-limiting) exemplary labeling moieties that may be used for, among other things, cancer detection and/or treatment. Certain moieties may be used for both detection and treatment. For example, certain radioreactive groups may be used as imaging agents when provided at low dose, but may also be used as therapeutic agents when provided at high dose.

In one embodiment, the functional moiety (or labeling moiety) is an affinity moiety, and thus the reagent is an affinity reagent. The affinity reagent has at least one affinity moiety that can be used to purify labeled proteins. In one embodiment, the affinity group is a biotin moiety. In another embodiment, the affinity group is an immunological or epitope tag. In a preferred embodiment, the affinity reagent has two or more affinity groups, thus allowing tandem purification of the labeled proteins. Tandem purification decreases contamination by proteins that non-specifically adhere to the matrices or support material (such as resin) used for purification.

The affinity reagent can have one or more cleavable sites that allow for release of the purified proteins from the affinity matrix. The cleavable sites can be peptide bonds cleavable by proteases, or can be chemical cleavage or photo-labile sites built into the affinity reagent. In a preferred embodiment, the cleavage sites are peptide sequences that can be specifically cleaved by proteases such as trypsin.

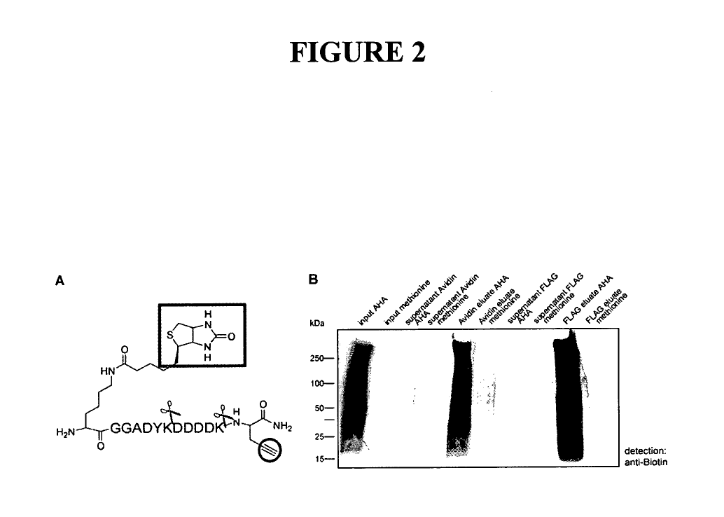

In certain embodiments, the cleavable sites are at or adjacent to the epitope tag. For example, in FIG. 2, the FLAG tag sequence contains two trypsin cleavage sites that allow for the cleavage of the FLAG sequence, thereby releasing the attached protein or fragment incorporating the non-natural amino acid.

Other exemplary functional moieties include, but are not necessarily limited to, fluorescent molecules or tags (e.g., auto-fluorescent molecules, molecules that fluoresce upon contact with a reagent, etc.), radioactive labels (e.g., ¹¹¹In, ¹²⁵I, ¹³¹I, ²¹²B, ⁹⁰Y, ¹⁸⁶Rh, and the like); biotin (e.g., to be detected through reaction of biotin and avidin); imaging reagents (e.g., those described in U.S. Pat. No. 4,741,900 and U.S. Pat. No. 5,326,856), and the like. Functional moieties may also include peptides or polypeptides that can be detected by antibody binding, e.g., by binding of a detectably labeled antibody or by detection of bound antibody through a sandwich-type assay.

In one embodiment, an engineered phosphine may act as an azide-selective phosphine dye. In this embodiment, the dye remains substantially undetectable until reaction with an azide. In general, the phosphorous lone pair renders the dye substantially undetectable (e.g., substantially non-fluorescent) until reaction with an azide, when the lone pair is removed from conjugation by formation of a phosphine oxide. The unmasked dye provides for a detectable signal following reaction with the azide of the target molecule according to the invention. This reaction is described in detail in paragraphs [0078]-[0085] of US 2002-0016003 A1 (incorporated herein by reference). Briefly, the masked dye generally comprises an aryl group substituted with R₁ and PR₂R₃; wherein R₁ is an electrophonic group to trap (e.g., stabilize) an aza-ylide group, including, but not necessarily limited to, a carboxylic acid, an ester (e.g., alkyl ester (e.g., lower alkyl ester, benzyl ester), aryl ester, substituted aryl ester), aldehyde, amide, e.g., alkyl amide (e.g., lower alkyl amide), aryl amide, alkyl halide (e.g., lower alkyl halide), hoister, colony ester, alkyl ketone (e.g., lower alkyl ketone), aryl ketone, substituted aryl ketone, halosulfonyl, nitrile, nitro and the like; R₂ and R₃ are generally aryl groups, including substituted aryl groups, or cycloalkyl groups (e.g., cyclohexyl groups) where R₂ and R₃ may be the same or different, preferably the same; R₁ and PR₂R₃ are preferably in the ortho position relative to one another on an aryl ring of the dye, or in an equivalent position that provides for the reaction to proceed according to the invention; X represents a target molecule having an azide N3; and where R₁′ represents a modified R₁ group following reaction with the azide of the target molecule via the Staudinger ligation reaction described herein.

In one exemplary embodiment, the phosphine dye is a fluorescein derivative, which may be in unacetylated or acetylated (cell permeable) form. Three phosphine dyes are described in detail in paragraphs [0086]-[0089] of US 2002-0016003 A1 (incorporated herein by reference). These phosphine dyes can be used to detect an azide on any molecule, such as those proteins incorporating AHA. The phosphine dyes can be used to detected biomolecules having an azide either at the cell surface or within cells.

(C) Cancer Killing Labeling Moieties

In certain embodiments, such as when the labeling moiety is a cytotoxic moiety, the labeling moiety may be linked to the second reactive group via a cleavable bond, such as a hydrolyzable bond that may be provided by use of an amide or ester group linking the cytotoxic moiety to the rest of the molecule.

In certain embodiments, the labeling moiety is a chelate moiety for chelating a metal, e.g., a chelator for a radiometal or paramagnetic ion. In certain embodiments, the labeling moiety is a chelator for a radionuclide useful for radiotherapy or imaging procedures. Radionuclides useful within the present invention include gamma-emitters, positron-emitters, Auger electron-emitters, X-ray emitters and fluorescence-emitters, with beta- or alpha-emitters (preferred for therapeutic use). Examples of radionuclides useful as toxins in radiation therapy include: ³²P, ³³P, ⁴³K, ⁴⁷Sc, ⁵²Fe, ⁵⁷Co, ⁶⁴Cu, ⁶⁷Ga, ⁶⁷Cu, ⁶⁸Ga, ⁷¹Ge, ⁷⁵Br, ⁷⁶Br, ⁷⁷Br, ⁷⁷As, ⁷⁷Br, ⁸¹Rb/^{81m}Kr, ^{87m}Sr, ⁹⁰Y, ⁹⁷Ru, ⁹⁹Tc, ¹⁰⁰Pd, ¹⁰¹Rh, ¹⁰³Pd, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹_{Os,} ¹⁹³Pt, ¹⁹⁴Ir, ¹⁹⁷Hg, ¹⁹⁹Au, ²⁰³Pb, ²¹¹At, ²¹²Pb, ²¹²Bi, and ²¹³Bi. Preferred therapeutic radionuclides include ¹⁸⁸Re, ¹⁸⁶Re, ²⁰³Pb, ²¹²Pb, ²¹²Bi, ¹⁰⁹Pd, ⁶⁴Cu, ⁶⁷Cu, ⁹⁰Y, ¹²⁵I, ⁹⁷Ru, ¹⁰⁵Rh, ¹⁹⁸Au, and ¹⁹⁹Ag, ¹⁶⁶Ho, or ¹⁷⁷Lu. Conditions under which a chelator will coordinate a metal are described, for example, by Gansow et al., U.S. Pat. Nos. 4,831,175, 4,454,106 and 4,472,509 (all incorporated herein by reference).

^{99m}Tc is a particularly attractive radioisotope for therapeutic and diagnostic applications, as it is readily available to all nuclear medicine departments, is inexpensive, gives minimal patient radiation doses, and has ideal nuclear imaging properties. It has a half-life of six hours, which means that rapid targeting of a technetium-labeled molecule (e.g., antibody or labeling reagent) is desirable. Accordingly, in certain preferred embodiments, the labeling reagent includes a chelating agents for technium.

The subject labeling reagent can also comprise a radio-sensitizing agents, e.g., a moiety that increase the sensitivity of cells to radiation. Examples of radio-sensitizing agents include nitroimidazoles, metronidazole and misonidazole (see: DeVita, V. T. Jr. in Harrison's Principles of Internal Medicine, p. 68, McGraw-Hill Hill Book Co., New York 1983, all incorporated herein by reference). The subject labeling reagent that comprises a radio-sensitizing agent as the active labeling moiety may be, as many other labeling reagents, systemically or locally administered and localizes at the metastasized cell. Upon exposure of the individual to radiation, the radio-sensitizing agent is “excited,” and causes cell death.

There are a wide range of moieties which can serve as chelating ligands and which can be derivatized to the labeling reagents. For instance, the chelating ligand can be a derivative of 1,4,7,10-tetraazacyclododecanetetraacetic acid (DOTA), ethylenediaminetetraacetic acid (EDTA), diethylenetriaminepentaacetic acid (DTPA) and 1-p-Isothiocyanato-benzyl-methyl-diethylenetriaminepentaacetic acid (ITC-MX). These chelators typically have groups on the side chain by which the chelator can be used for attachment to the subject labeling reagent. Such groups include, e.g., benzylisothiocyanate, by which the DOTA, DTPA or EDTA can be coupled to, e.g., an amine group on the subject labeling reagent.

In one embodiment, the subject labeling moiety is an “NₓS_{y}” chelate moiety. As defined herein, the term “NₓS_{y} chelates” includes bifunctional chelators that are capable of coordinately binding a metal or radiometal and, preferably, have N₂S₂ or N₃S cores. Exemplary NₓS_{y} chelates are described, e.g., in Fritzberg et al. (1988) PNAS 85: 4024-29; and Weber et al. (1990) Bioconjugate Chem. 1: 431-37; and in the references cited therein.

The Jacobsen et al. PCT application WO 98/12156 provides methods and compositions, i.e., synthetic libraries of binding moieties, for identifying compounds which bind to a metal atom. The approach described in that publication can be used to identify binding moieties which can subsequently be added to the subject labeling reagent.

A problem frequently encountered with the use of conjugated proteins in radio-therapeutic and radiodiagnostic applications is a potentially dangerous accumulation of the radiolabeled moiety fragments in the kidney. When the conjugate is formed using a acid- or base-labile linker, cleavage of the radioactive chelate from the protein can advantageously occur. If the chelate is of relatively low molecular weight, as most of the subject labeling reagent are expected to be, it is not retained in the kidney and is excreted in the urine, thereby reducing the exposure of the kidney to radioactivity. However, in certain instances, it may be advantageous to utilize acid- or base-labile linkers in the subject labeling reagent for the same reasons they have been used in labeled proteins.

Accordingly, certain linkers of the subject labeling moiety can be synthesized, by standard methods known in the art, to provide reactive functional groups which can form acid-labile linkages with, e.g., a carbonyl group of the subject labeling reagent. Examples of suitable acid-labile linkages include hydrazone and thiosemicarbazone functions. These are formed by reacting the oxidized carbohydrate with chelates bearing hydrazide, thiosemicarbazide, and thiocarbazide functions, respectively.

Alternatively, base-cleavable linkers, which have been used for the enhanced clearance of the radiolabel from the kidneys, can be used. See, for example, Weber et al. 1990 Bioconjug. Chem. 1: 431. The coupling of a bifunctional chelate to a subject labeling reagent via a hydrazide linkage can incorporate base-sensitive ester moieties in a linker spacer arm. Such an ester-containing linker unit is exemplified by ethylene glycolbis(succinimidyl succinate), (EGS, available from Pierce Chemical Co., Rockford, Ill.), which has two terminal N-hydroxysucciuimide (NHS) ester derivatives of two 1,4-dibutyric acid units, each of which are linked to a single ethylene glycol moiety by two alkyl esters. One NHS ester may be replaced with a suitable amine-containing BFC (for example 2-aminobenzyl DTPA), while the other NHS ester is reacted with a limiting amount of hydrazine. The resulting hyrazide is used for coupling to the subject labeling reagent, forming an ligand-BFC linkage containing two alkyl ester functions. Such a conjugate is stable at physiological pH, but readily cleaved at basic pH.

See the next section for additional cleavable linkages that may be used with the subject labeling reagent.

The subject labeling reagent labeled by chelation are subject to radiation-induced scission of the chelator and to loss of radioisotope by dissociation of the coordination complex. In some instances, metal dissociated from the complex can be re-complexed, providing more rapid clearance of non-specifically localized isotope and therefore less toxicity to non-target tissues. For example, chelator compounds such as EDTA or DTPA can be infused into patients to provide a pool of chelator to bind released radiometal and facilitate excretion of free radioisotope in the urine.

In still other embodiments, the subject labeling reagent is a Boron addend, such as a carborane. For example, carboranes can be prepared with carboxyl functions on pendant side chains, as is well known in the art. Attachment of such carboranes to an amine functionality, e.g., as may be provided on the subject labeling reagent, can be achieved by activation of the carboxyl groups of the carboranes and condensation with the amine group to produce the conjugate. Such modified labeling reagent can be used for neutron capture therapy.

In still other embodiments, the subject labeling reagent includes a cytotoxic moiety as the labeling moiety, such as a chemotherapeutic agent or a toxin. Many drugs and toxins are known which have cytotoxic effects on cells, and can be used in connection with the present invention. They are to be found in compendia of drugs and toxins, such as the Merck Index, Goodman and Gilman, and the like, and in the references cited above.

Chemotherapeutics useful as active moieties when conjugated to the subject labeling reagent may be specifically delivered to primary and/or metastasized cancers. Such chemotherapeutics are typically small chemical entities produced by chemical synthesis. Chemotherapeutics include cytotoxic and cytostatic drugs. Chemotherapeutics may include those which have other effects on cells such as reversal of the transformed state to a differentiated state or those which inhibit cell replication.

Examples of known cytotoxic agents useful in the present invention are listed, for example, in Goodman et al., “The Pharmacological Basis of Therapeutics,” Sixth Edition, A. G. Gilman et al., eds./Macmillan Publishing Co. New York, 1980. These include taxol, nitrogen mustards, such as mechlorethamine, cyclophosphamide, melphalan, uracil mustard and chlorambucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be used; such drugs are known to these skilled in the art and include puromycin, cycloheximide, and ribonuclease.

Prodrug forms of the chemotherapeutic moiety are especially useful in the present invention to generate an inactive precursor.

Most of the chemotherapeutic agents currently in use in treating cancer possess functional groups that are amenable to chemical crosslinking directly with an amine or carboxyl group of a subject labeling reagent. For example, free amino groups are available on methotrexate, doxorubicin, daunorubicin, cytosinarabinoside, cis-platin, vindesine, mitomycin and bleomycin, while free carboxylic acid groups are available on methotrexate, melphalan, and chlorambucil. These functional groups, that is free amino and carboxylic acids, are targets for a variety of homobifunctional and heterobifunctional chemical crosslinking agents which can crosslink these drugs directly to a free amino group of a subject labeling reagent.

Peptide and polypeptide toxins are also useful as active moieties, and the present invention specifically contemplates embodiments wherein the labeling moiety comprises a toxin. Toxins are generally complex toxic products of various organisms including bacteria, plants, etc. Examples of toxins include but are not limited to: ricin, ricin A chain (ricin toxin), Pseudomonas exotoxin (PE), Diphtheria toxin (DT), Clostridium perfringens phospholipase C(PLC), bovine pancreatic ribonuclease (1BPR), pokeweed antiviral protein (PAP), abrin, abrin A chain (abrin toxin), cobra venom factor (CVF), gelonin (GEL), saporin (SAP), modeccin, viscumin and volkensin.

In addition, there are other active agents which can be used to create a subject labeling reagent for the treatment of cancer. For example, the subject labeling reagent can be generated to include active enzyme. The subject labeling reagent specifically localizes the activity to the tumor cells. An inactive prodrug which can be converted by the enzyme into an active drug may be administered to the patient. The prodrug is only converted to an active drug by the enzyme which is localized to the tumor (e.g., by localized delivery to the tumor). An example of an enzyme/prodrug pair includes alkaline phosphatase/etoposidephosphate. In such a case, the alkaline phosphatase is conjugated to a subject labeling reagent. The subject labeling reagent is then administered and localizes at the metastasized cell. Upon contact with etoposidephosphate (the prodrug), the etoposidephosphate is converted to etoposide, a chemotherapeutic drug which is taken up by the cancer cell.

The present invention also contemplates dyes used, for example, in photodynamic therapy, and used in conjunction with appropriate non-ionizing radiation. The use of light and porphyrins in methods of the present invention is also contemplated and their use in cancer therapy has been reviewed, van den Bergh, Chemistry in Britain, 22: 430-437 (1986), which is incorporated herein in its entirety by reference.

(D) Cleavable Functional Group

The reagents with the second reactive moiety of the invention may optionally contain one or more cleavable functional groups to allow separation of the second reactive moiety and the functional moiety under one or more controlled conditions.

As used herein, a “cleavable functional group” or “cleavable linker” is a chemical group that can be cleaved by a variety of methods, including input of energy, a chemical, an enzyme, and the like. For use in methods of the invention, the cleavable functional group is generally specific, that is, one which can be specifically cleaved without altering or damaging the molecule being cleaved or which relatively uniformly alters the molecule in a reproducible manner. For example, the cleavable functional group can be a photo-cleavable group. In such a case, the photo-cleavable group is generally cleaved at a wavelength of light that does not damage the molecule being released, for example, in the ultraviolet to visible range. Exemplary photo-cleavable linkers include, for example, linkers containing o-nitrobenzyl, desyl, trans-cinnamoyl, m-nitrophenyl, benzylsulfonyl groups and the like (see, for example, Dorman and Prestwich, Trends Biotech. 18: 64-77, 2000; Greene and Buts, “Protective Groups,” in Organic Synthesis, 2nd ed., John Wiley & Sons, New York, 1991; U.S. Pat. Nos. 5,143,854; 5,986,076; 5,917,016; 5,489,678; 5,405,783).

The cleavable functional group can also be a chemically cleavable group cleavable by a chemical such as an acid or base. If desired, a chemically cleavage reaction can be carried out under relatively mild conditions in which the chemically cleavable group is essentially the only chemical bond cleaved. A chemically cleavable group can also be a group cleavable by a chemical such as CNBr, which can cleave a methionine residue. CNBr can be particularly useful for releasing a molecule if a chemically cleavable group such as methionine has been added to the molecule, particularly in a polypeptide that does not have a methionine residue, or when the methionine residues have been replaced by non-natural amino acids.

Suitable chemically cleavable groups are well known to those skilled in the art (see, for example Wilson and Czarnik, eds., Combinatorial Chemistry: Synthesis and Application, John Wiley & Sons, New York, 1997; Merrifield, J. Am. Chem. Soc. 85: 2149, 1964; Bodanszky, Principles of Peptide Synthesis, Springer-Verlag, 1984; Houghten, Proc. Natl. Acad. Sci., USA 82: 5131, 1985). Exemplary chemically cleavable linkers can contain a disulfide, which can be cleaved with reducing agents; a diol, which can be cleaved with periodate; a diazo bond, which can be cleaved with dithionate; an ester, which can be cleaved with hydroxylamine; a sulfone, which can be cleaved with base, and the like (see Hermanson, Bioconjuqate Techniques, Academic Press, San Diego, 1996; Pierce Chemical, Rockford Il.).

The cleavable functional group can also be an enzymatically cleavable group. For example, a protease can be used to cleave a cleavable functional group having a suitable recognition sequence for the protease.

Particularly useful proteases are endopeptidases such as factor Xa, tobacco etch virus (TEV) protease, trypsin, chymotrypsin, Staphylococcus aureus protease, submaxillaris protease, and the like. The protease can be selected based on the incorporation of a particular cleavable recognition sequence as a functional group. Other considerations for selecting a protease include the presence or absence of a recognition sequence in the molecule being captured and released. For example, a rare cleaving protease such as TEV protease or factor Xa can be used to cleave a functional group containing the corresponding protease recognition sequence, resulting in release of the captured molecule.

Such rare cleaving proteases are particularly useful for releasing an intact polypeptide molecule since the recognition sequence for these proteases would not occur in the vast majority of polypeptides. Alternatively, a polypeptide sample can be treated with a specific protease, and the digested peptides isolated by the methods disclosed herein. In such a case, the captured peptides would not contain a recognition sequence for the protease used for cleavage since the polypeptide has already been digested.

In addition, in certain embodiments, an intact polypeptide can be captured and digested with a protease after binding to the solid support, resulting in the incorporation and release of a label on the peptide fragment of the polypeptide that was captured on the solid support. Thus, protease digestion can be used before or after capture of a sample molecule, in particular polypeptide sample molecules, as desired.

In addition to proteases, a cleavable functional group can be a recognition sequence for an endonuclease such as a restriction enzyme. Thus, an appropriate recognition sequence for a restriction enzyme can be incorporated as a cleavable functional group and cleaved with the respective restriction enzyme. It is understood that such a nucleotide functional group can be useful for capturing and releasing a nucleic acid or a polypeptide, or any other type of molecule, as desired. Similarly, a protease recognition sequence can be useful for capturing and releasing a polypeptide, nucleic acid or any other type of molecule, as desired.

As used herein, the term “isotopic label” or “isotope tag” refers to a chemical group which can be generated in two distinct isotopic forms, for example, heavy and light isotopic versions of the constituent elements making up the chemical group. Such constituent elements include, for example, carbon (e.g., ¹³C or ¹⁴C), oxygen (e.g., ¹⁸O) hydrogen (e.g., ²H or ³H), nitrogen (e.g., ¹⁵N), and sulfur (e.g., ³⁵S). In addition, other elements that are chemically or functionally similar can be substituted for the above naturally occurring elements. For example, selenium can be used as a substitute for sulfur. Particularly useful isotopic labels or tags are those that allow convenient analysis by MS. In such cases, the isotope is preferably non-radioactive or with low radioactivity. For example, heavy and light isotopic versions of an amino acid can be used to differentially isotopically label a polypeptide.

### 4. Detection and Analysis

After tryptic digestion, mass spectral analysis may be used to analyze the eluted non-natural amino acid-containing proteins or fragments thereof.

In one embodiment, the eluted proteins may be analyzed using conventional techniques, such as 2-D electrophoresis.

In a preferred embodiment, detection and analysis is achieved by utilizing MudPIT (Multi-dimensional Protein Identification Technology) or related advanced mass spectrometrical techniques. MudPIT is a technique for the separation and identification of complex protein and peptide mixtures. MudPIT is another approach developed to alleviate many of the disadvantages associated with two-dimensional gel electrophoresis. MudPIT uses two chromatography steps interfaced back to back in a fused silica capillary. More specifically, MudPIT uses columns consisting of strong cation exchange (SCX) material back-to-back with reversed phase (RP) material inside fused silica capillaries. The chromatography proceeds in cycles, each comprising an increase in salt concentration to “bump” peptides off of the SCX followed by a gradient of increasing hydrophobicity to progressively elute peptides from the RP into the ion source. In other words, chromatography proceeds in steps with increases in salt concentration used to free peptides from the cation-exchange resin, after which they bind to a reversed phase resin. A typical reversed phase gradient to increasing hydrophobicity is then applied to progressively elute peptides from the reversed phase packing into the mass spectrometer. Typically, this mass spectrometer will be a tandem electrospray (ESI-MS/MS), so peptides are ionized in the liquid phase, separated in a primary mass spectrometer, broken up using collision-induced dissociation (CID) and analyzed again.

The advantage of this process is that the band broadening associated with many chromatographic steps is avoided, thus avoiding loss of resolution, and preventing components from running into one another. In addition, the capillary can be placed directly into the ion source of a mass spectrometer to maximize sensitivity.

Raw mass spectral data are filtered and analyzed using art-recognized methods, such as SEQUEST, DTASelect and Contrast algorithms, which allow for an efficient and comprehensive interpretation and comparison of the proteomic data. Specifically, the mass spectrometer's data-dependent acquisition isolates peptides as they elute, and subjects them to Collision-Induced Dissociation, recording the fragment ions in a tandem mass spectrum. These spectra are matched to database peptide sequences by the SEQUEST algorithm. SEQUEST's peptide identifications are assembled and filtered into protein-level information by the DTASelect algorithm.

In a typical setting, a Sutter Instrument Company P-2000 is used to produce tips from fused silica capillaries, which are usually purchased from Polymicro or Agilent. Apertures are approximately 5 nanometers across from capillaries that have an inner diameter of 100 microns. The material that is loaded into the SCX columns can be made from 5 micron spherical silica beads. For RP, 5 micron C-18 coated beads from a variety of commercial vendors can be used. Agilent 1100 and ThermoFinnigan Surveyor Quaternary pumps may be operated at flow rates of 100-200 microliters/min, with pre-column splitting of the flow to produce about 100-200 mL/min flow rates at the column.

SEQUEST is a program that correlates uninterpreted tandem mass spectra of peptides with amino acid sequences from protein and nucleotide databases. SEQUEST will determine the amino acid sequence and thus the protein(s) and organism(s) that correspond to the mass spectrum being analyzed. SEQUEST is distributed by Thermo Finnigan, A division of Thermo Electron Corporation. SEQUEST uses algorithms described in U.S. Pat. Nos. 6,017,693 and 5,538,897.

While SEQUEST is very powerful for matching uninterpreted tandem mass spectra to database peptide sequences, DTASelect is designed to reassemble this peptide information into usable protein information. The DTASelect program can do more than simply report the protein content of a sample; it features many customizable filters to specify which identifications should be kept and which discarded. It also features reports to investigate post-translational modifications, align sequences of peptides to identify poorly sequenced regions, and analyze chromatography efficiency. The software makes the process of analyzing SEQUEST results much faster, more powerful, and more consistent than possible before, even for data sets containing a million spectra or more. By automating SEQUEST analysis, DTASelect enables experiments of far greater scope. Alternatively, programs like PeptideProphet and ProteinProphet can be used for data processing after the SEQUEST algorithm. Programs mentioned above and in the following paragraph are only examples for performing the data analysis.

Differentiating biological samples by protein content is an important application of proteomics. The CONTRAST program uses the filters present in DTASelect to highlight the most important identifications of samples and then compares them. Unlike most relatively simple comparison algorithms, Contrast can differentiate up to 63 different samples at once. Contrast handles differential analysis between experimental and control samples simply and flexibly.

Both the DTASelect and the Contrast programs can be licensed for use from The Scripps Research Institute (La Jolla, Calif.). For reference, see Tabb et al., DTASelect and Contrast: Tools for Assembling and Comparing Protein Identifications from Shotgun Proteomics. J. Proteome Res. 1: 21-26, 2002 (incorporated herein by reference).

### 5. Pharmaceutical Compositions and Administration

The subject labeling reagent and/or non-natural amino acid can be formulated as pharmaceutical compositions for human or veterinary use. For example, they may be formulated as a solution, suspension or emulsion in association with a pharmaceutically acceptable parenteral vehicle. Examples of such vehicles are water, saline, Ringer's solution, dextrose solution, and 5% human serum albumin. Liposomes may also be used. The vehicle may contain additives that maintain isotonicity (e.g., sodium chloride, mannitol) and chemical stability (e.g., buffers and preservatives). The formulation is sterilized by commonly used techniques. For example, a parenteral composition suitable for administration by injection is prepared by dissolving 1.5% by weight of active ingredient in 0.9% sodium chloride solution.

The pharmaceutical compositions according to the present invention may be administered as either a single dose or in multiple doses. Individual components of a pharmaceutical composition (e.g., the subject non-natural amino acid(s) and the subject labeling reagent) may be formulated together or separately, for simultaneous or sequential administration to the patient.

The pharmaceutical compositions of the present invention may be administered either as individual therapeutic agents or in combination with other therapeutic agents. The treatments of the present invention may be combined with conventional therapies, which may be administered sequentially or simultaneously. The pharmaceutical compositions of the present invention may be administered by any appropriate means or combinations thereof that enable them to reach the targeted cells. In some embodiments, routes of administration include those selected from the group consisting of oral, transdermal, topical, intramuscular (i.m.), intravenous (i.v.), intraarterial, intraperitoneal (i.p.), and/or local administration into the blood supply of the organ in which the tumor resides or directly into the tumor itself. In certain embodiments, intravenous administration is the preferred mode of administration. It may be accomplished with the aid of an infusion pump.

The dosage administered varies depending upon factors such as: the nature of the active moiety, the nature of the subject non-natural amino acid or the labeling reagent; their respective pharmacodynamic characteristics; its mode and route of administration; age, gender, health, and weight of the recipient; types of cancer or disease treated; nature and extent of symptoms; kind of concurrent treatment; and frequency of treatment.

Because the subject ligands are specifically targeted to cells preferentially incorporating the subject non-natural amino acids, the subject labeling reagent (with the labeling moiety), which comprise chemotherapeutics or toxins, are administered in doses less than those which are used when the same chemotherapeutics or toxins are administered as unconjugated active agents, preferably in doses that contain up to 100 times less active agent. In some embodiments, the subject labeling reagent which comprises chemotherapeutics or toxins are administered in doses that contain 10-100 times less active agent as an active moiety than the dosage of chemotherapeutics or toxins administered as unconjugated active agents. To determine the appropriate dose, the amount of compound is preferably measured in moles instead of by weight. In that way, the variable weight of different labeling reagent does not affect the calculation. Presuming a one to one ratio of the subject labeling reagent to active moiety in the subject labeling reagent, less moles of the subject labeling reagent may be administered as compared to the moles of unmodified labeling moieties administered, preferably up to 100 times less moles.

### 6. Exemplary Uses

A. Treatment of Disease Conditions

The present invention relates to a method of detecting and/or treating a cancer or a hyper-proliferative disease in a human or a non-human animal. In theory, any cancer or undesirable hyper-proliferative disease (including non-cancer) may be detected and/or treated with the subject compositions and methods.

For example, a non-limiting list of cancer that may be treated with the reagents of the present invention include various solid tumors, sarcoma, or cancers of the blood, such as: ACTH-producing tumors, acute lymphocytic leukemia, acute nonlymphocytic leukemia, cancer of the adrenal cortex, bladder cancer, brain cancer, breast cancer, cervix cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head & neck cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovary cancer, ovary (germ cell) cancer, pancreatic cancer, penis cancer, retinoblastoma, skin cancer, soft-tissue sarcoma, squamous cell carcinomas, stomach cancer, testicular cancer, thyroid cancer, trophoblastic neoplasms, cancer of the uterus, vaginal cancer, cancer of the vulva and Wilm's tumor.

For patients who initially present without advanced or metastatic cancer, the subject labeling reagent-based drugs are used as an immediate initial therapy prior to surgery and radiation therapy, and as a continuous post-treatment therapy in patients at risk for recurrence or metastasis (based upon high Gleason's score, locally extensive disease, and/or pathological evidence of tumor invasion in the surgical specimen, etc.). The goal in these patients is to inhibit the growth of potentially metastatic cells from the primary tumor during surgery or radiotherapy and inhibit the growth of tumor cells from undetectable residual primary tumor.

For patients who initially present with advanced or metastatic cancer, the subject labeling reagent-based drugs are used as a continuous supplement to, or possible as a replacement for hormonal ablation. The goal in these patients is to slow tumor cell growth from both the untreated primary tumor and from the existing metastatic lesions.

In addition, the invention may be particularly efficacious during post-surgical recovery, where the present compositions and methods may be particularly effective in lessening the chances of recurrence of a tumor engendered by shed cells that cannot be removed by surgical intervention.

The present invention also includes a diagnostic kit for performing the methods of the present invention and may contain compounds and/or compositions containing the compounds of the present invention. For instance, radiolabeled ligands or fluorescent moieties may be used in a manner so as to provide diagnostic information. Examples of diagnostic information and uses include determining the type of disease, the progress of the particular disease, the location of cells targeted by the subject labeling reagents and similar diagnostic uses known to persons skilled in the art.

In the methods of the present invention, the compounds may be administered orally, parenterally, by inhalation spray, topically, rectally, nasally, buccally, vaginally or via an implanted reservoir in dosage formulations containing conventional non-toxic pharmaceutically-acceptable carriers, adjuvants and vehicles. The term parenteral as used herein includes subcutaneous, intravenous, intramuscular, intraperitoneal, intrathecal, intraventricular, intrasternal or intracranial injection and infusion techniques. Invasive techniques may be preferred, particularly direct administration to damaged/cancerous tissue.

In certain embodiments, to be effective therapeutically for central nervous system targets, such as various brain cancer, the compounds of the present invention should readily penetrate the blood-brain barrier when peripherally administered. Compounds which cannot penetrate the blood-brain barrier can be effectively administered by an intraventricular route.

The compounds may also be administered in the form of sterile injectable preparations, for example, as sterile injectable aqueous or oleaginous suspensions. These suspensions can be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile fixed oils are conventionally employed as solvents or suspending mediums. For this purpose, any bland fixed oil such as a synthetic mono- or di-glyceride may be employed. Fatty acids such as oleic acid and its glyceride derivatives, including olive oil and castor oil, especially in their polyoxyethylated forms, are useful in the preparation of injectables. These oil solutions or suspensions may also contain long-chain alcohol diluents or dispersants.

Additionally, the compounds may be administered orally in the form of capsules, tablets, aqueous suspensions or solutions. Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening and/or flavoring and/or coloring agents.

The compounds may further be administered rectally in the form of suppositories. These compositions can be prepared by mixing the drug with suitable non-irritating excipients which are solid at room temperature, but liquid at rectal temperature such that they will melt in the rectum to release the drug. Such excipients include cocoa butter, beeswax and polyethylene glycols.

Moreover, the compounds may be administered topically, especially when the conditions addressed for treatment involve areas or organs readily accessible by topical application, including neurological disorders of the eye, the skin (e.g., melanoma) or the lower intestinal tract.

For topical application to the eye, or ophthalmic use, the compounds can be formulated as micronized suspensions in isotonic, pH adjusted sterile saline or, preferably, as a solution in isotonic, pH adjusted sterile saline, either with or without a preservative such as benzylalkonium chloride. Alternatively, the compounds may be formulated into ointments, such as petrolatum.

For topical application to the skin, the compounds can be formulated into suitable ointments containing the compounds suspended or dissolved in, for example, mixtures with one or more of the following: mineral oil, liquid petrolatum, white petrolatum, propylene glycol, polyoxyethylene polyoxypropylene compound, emulsifying wax and water. Alternatively, the compounds can be formulated into suitable lotions or creams containing the active compound suspended or dissolved in, for example, a mixture of one or more of the following: mineral oil, sorbitan monostearate, polysorbate 60, cetyl ester wax, cetearyl alcohol, 2-octyldodecanol, benzyl alcohol and water.

Topical application to the lower intestinal tract can be effected in rectal suppository formulations (see above) or in suitable enema formulations.

The compounds of the present invention may be administered by a single dose, multiple discrete doses or continuous infusion. Since the compounds are small, easily diffusible and relatively stable, they are well suited to continuous infusion. Pump means, particularly subcutaneous pump means, are preferred for continuous infusion.

Compositions and methods of the invention may also use controlled release technology. For example, the subject labeling reagents or non-natural amino acids may be incorporated into a polymer matrix for controlled release over a period of days. Such controlled release films are well known to the art. Examples of polymers commonly employed for this purpose that may be used in the present invention include nondegradable ethylene-vinyl acetate copolymer and degradable lactic acid-glycolic acid copolymers. Certain hydrogels such as poly(hydroxyethylmethacrylate) or poly(vinylalcohol) also may be useful.

Dose levels on the order of about 0.1 mg to about 10,000 mg of the active ingredient compound are useful in the treatment of the above conditions, with preferred levels being about 0.1 mg to about 1,000 mg. The specific dose level for any particular patient will vary depending upon a variety of factors, including the activity of the specific compound employed; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models are also helpful, particularly in determining effective doses for treating cancer. The considerations for determining the proper dose levels are well known in the art.

For the methods of the present invention, any administration regimen regulating the timing and sequence of drug delivery can be used and repeated as necessary to effect treatment. Such regimen may include pretreatment and/or co-administration with additional therapeutic agents.

For patients with cancer that is neither advanced nor metastatic, the compounds of the present invention may be administered (i) prior to surgery or radiation treatment to reduce the risk of metastasis; (ii) during surgery or in conjunction with radiation treatment; and/or (iii) after surgery or radiation therapy to reduce the risk of recurrence and to inhibit the growth of any residual tumorous cells.

For patients with advanced or metastatic cancer, the compounds of the present invention may be administered as a continuous supplement to, or as a replacement for, hormonal ablation in order to slow tumor cell growth in both the untreated primary tumor and the existing metastatic lesions.

The methods of the present invention are particularly useful where shed cells could not be removed by surgical intervention. After post-surgical recovery, the methods of the present invention would be effective in reducing the chances of recurrence of a tumor engendered by such shed cells.

The instant invention can also be used to determine the abundance of newly synthesized proteins in a sample, based on the ability of certain labeling moiety (such as affinity moieties) to bind affinity reagents. For example, the sample (such as cell or tissue lysates) containing non-natural amino acids labeled by the subject labeling moieties may be incubated with one or more affinity reagents that bind the labeling moiety to allow binding and isolation/purification of the labeled proteins. Bound proteins can then be separated from unbound (unlabeled) ones, and the abundance of the bound labeled proteins can be determined conveniently, especially when the abundance of the modifying group (e.g., the labeling moiety) is to be measured by, for example, mass spectrometry (supra).

The amount of bound protein is partly determined by the reaction efficiency between the reactive groups, it is also determined by the amount of labeled protein in the sample. The relative amount of labeled proteins within different samples can be compared directly. However, when an absolute amount of labeled proteins within a sample is desired, a series of control samples with known labeled protein concentrations can be employed to derive a standard curve, from which the absolute concentration of the labeled proteins in a test sample can be deduced.

There are many ways to determine the abundance of the labeled proteins. For example, if the modifying group is a fluorescent tag, the amount of fluorescent signals is a measurement of the label abundance. Similarly, the amount of chelate groups can be determined by the amount of radio-isotopes that can be bound by the chelate groups. Other embodiments of the invention will be apparent to skilled artisans.

Separation of bound and unbound proteins can be achieved in many conceivable ways. For example, affinity reagents within a test sample can be fixed on a solid support, such as in a well of a microtiter plate. Excessive unbound proteins, just like in an ELISA assay, can then be washed away. Alternatively, the labeling moiety can be immunoprecipitated using a specific antibody, and the amount of the associated labels determined using the methods described before.

B. Combination with Other Treatments

(i) Surgery and Radiation Treatment

In general, surgery and radiation treatment are employed as potentially curative therapies for patients with localized cancer that has not invaded and/or metastasized to the neighboring or remote tissues. However, a significant percentage of patients who have gone through the primary treatment will eventually develop recurrence within several years after such primary treatment. Thus there is considerable opportunity to use the present invention in conjunction with surgery and/or radiation treatment.

(ii) Hormonal Therapy

Hormonal ablation is an effective palliative treatment for certain patients with metastatic cancer, such as prostate cancer. Hormonal ablation by medication and/or orchiectomy is used to block hormones that promote further growth and metastasis of prostate cancer. With time, both the primary and metastatic tumors of virtually all of these patients may become hormone-independent and resistant to therapy. Thus continuous supplementation with the compounds of the present invention may be used to prevent or reverse this potentially metastasis-permissive state.

(iii) Chemotherapy

Chemotherapy has been successful in treating many forms of cancer, but it has shown less therapeutic value in treating other cancers where it is generally reserved as a last resort. However, when combined with the subject treatment, the chemotherapeutic agents may be much more effective due to the targeted delivery of such agents to cancer. Thus such treatments should be more effective than chemotherapy alone in controlling cancer.

(iv) Immunotherapy

The compounds of the present invention may also be used in combination with monoclonal antibodies to treat cancer.

The present invention may also be used with immunotherapies based on polyclonal or monoclonal antibody-derived reagents. Monoclonal antibody-derived reagents are preferred. These reagents are well known in the art, and include radiolabeled monoclonal antibodies such as monoclonal antibodies conjugated with strontium-89.

In certain embodiments, the antibody recognizes/binds a labeling moiety or an epitope tag on the labeling reagent, so as to specifically deliver the cancer killing agents to cancer.

(v) Cryotherapy

The methods of the present invention may also be used in conjunction with cryotherapy for treatment of cancer, such as prostate cancer.

C. Other Uses

The methods and reagents (such as kits) of the invention may also be used in research and development, such as labeling cells in vitro.

For example, to exclude contamination by somatically synthesized proteins, newly synthesized proteins from either synaptoneurosomes or isolated dendrites of hippocampal cultures can be analyzed. Isolated dendrites are obtained from a special culture system using polycarbonate nets to separate dendrites from cell bodies. Individual datasets of newly synthesized proteins can be obtained following different parameters of synaptic stimulation. Once compared, these datasets provide a detailed picture of the local molecular changes associated with different patterns of synaptic activity or the presence or absence of different neuromodulators. Furthermore, the approach allows the applicants to assess the general translational capacity of dendrites by quantifying single proteins in comparison to their ¹⁵N-substituted counterparts. Verification of the data can be examined at the mRNA level (in situ hybridization, RT-PCR) as well as at the protein level (immunocytochemistry and immunoprecipitation studies). The use of cell permeable, fluorescent alkyne-tags further expands this technology to in vivo real-time imaging of local protein synthesis in small dendritic segments by restricted application of labeling reagents and neuromodulators. This helps to address the question of spatial specificity of whether locally synthesized proteins are delivered only to the primary site of activation, or are they delivered to neighboring synapses as well.

Other possible applications, for instance, include proteomic profiling of transgenic animals in comparison to their wild-type littermates or profiling of pathological states and changes during development of a tissue and even a whole organism. Knowing the proteomic differences of two cellular compartments, like somata and dendrites, or a brain region challenged either by a missing particular gene or a neuropathological state, helps to understand the molecular and subcellular mechanisms of learning and memory formation, and of neurodegenerative diseases.

The invention circumvents the various problems associated with traditional approaches, by providing an approach to protein identification that unites techniques from organic chemistry, high throughput mass spectrometry and bioinformatics. For example, the procedure can be used to label newly synthesized dendritic proteins.

### EXAMPLES

The invention now being generally described, it will be more readily understood by reference to the following examples which are included merely for purposes of illustration of certain aspects and embodiments of the present invention, and are not intended to limit the scope of the invention.

### Example 1

### Culturing Neurons

Certain experiments described herein are conducted in neurons. This example provides an illustrative example for culturing neurons and the associated experimental conditions. Other art-recognized methods may also be used for substantially the same purpose.

Dissociated hippocampal neuron cultures were prepared from newborn rat pups (PO) as outlined in (Banker and Goslin, 1990), Neurons were plated at a density of 15,000-45,000 cells/cm² onto poly-D-lysine coated cell culture dishes or onto poly-D-lysine and growth factor reduced matrigel (BD Biosciences) coated polycarbonate nets with a pore size of 3 μm (Transwell, Corning) for the preparation of isolated dendrites. The cultures were maintained and allowed to mature in growth medium (Neurobasal-A supplemented with B27 and GlutaMAX-1) for 14 to 21 days before use. The use of this growth medium suppressed glial proliferation, which was even more reduced by application of Ara-C (5 μM final concentration). To supplement glial neurotrophic factors, 25% conditioned growth media from glial and cortical cultures were added to the growth media. Isolated dendrites were obtained from Transwell cultures by removing the cell body layer with a sterile cell lifter after change to HBS. For immunolabeling after AHA-incorporation and cycloaddition with BiotinCyclooctyne, neurons are fixed at room temperature with 4% paraformaldehyde for 20 minutes. Fixed cultures are then treated sequentially with PBS, blocking solution (0.2% Triton X-100, 2 mg/ml BSA, 5% Sucrose, 10% normal horse serum in PBS), primary Ab in blocking solution at 4° C. overnight, PBS-Tx (0.2% Triton X-100 in PBS), Alexa488- or Alexa568-conjugated secondary Ab in blocking solution, PBS-Tx and PBS. Immunostained specimens are imaged in PBS with an Olympus confocal microscope or two-photon microscope through either a 10× or a 63× oil-immersion lens. Alexa568 is excited at 543 nm and Alexa488 at 488 nm. Images are recorded through standard emission filters at contrast settings for which the crossover between the two channels is negligible.

### Example 2

### [3+2] Cycloaddition Chemistry and Purification of Tagged Proteins

In embodiments using [3+2] Cycloaddition, the following exemplary protocol may be used. Briefly, AHA, Biotin-PEO-Propargylamide and the triazole ligand were prepared as described previously [3, 46, 47]. The tandem featured alkyne tag was synthesized by GenScript Corporation. Biotin-Cyclooctyne is a generous gift from Carolyn Bertozzi [45]. D10-L-leucine was purchased from Sigma. In all culture experiments, growth medium was removed from HEK293 cells, whole neuronal cultures or isolated dendrites were replaced with HEPES-buffered solution (HBS) [48] with 2.86 mM AHA, and 2.86 mM methionine for control experiments. After incubation at 37° C., 5% CO₂, cells were washed with PBS-MC (1 mM MgCl₂, 0.1 mM CaCl₂ in PBS) to remove excess amounts of AHA and methionine. SNS and other biochemical fractions are incubated with 2.86 mM AHA or 2.86 mM methionine under agitation at 37° C. and washed in StimBuffer after incubation. Cells and fractions are lysed in 0.5% SDS, 1% Triton X-100 in PBS, with DNA destroyed by syringe and needle use. Lysates are diluted to 0.1% SDS, 0.2% Triton X-100 in PBS, complete protease inhibitor and treated for 10 min at 70° C. After addition of 200 μM triazole ligand, 50 μM alkyne tag and 75 μg/ml CuBr, the reaction is allowed to proceed for 16 hours at 4° C. After conclusion of the reaction, samples are dialyzed in 0.2% Triton X-100, 1 mM EDTA in PBS to remove excess reagents. Tagged proteins are purified using monomeric avidin columns followed by FLAG-M2-antibody immunoprecipitations. Eluted proteins are denatured in 8M Urea and proteo-lytically digested with endoproteinase Lys-C and trypsin (both Roche) as described in [49].

### Example 3

### Improved Cu(I) Catalysis

An aliquot of cells expressing recombinant OmpC (1 mL) was centrifuged at 4° C. and washed once in 1 mL of PBS (pH 7.4). The cells were centrifuged and resuspended in 1 mL of PBS. Triazole ligand 5 was added to a final concentration of 200 μM, and biotin-PEO-propargylamide 6 was added to a final concentration of 50 μM. Addition of the active copper species was accomplished in two different ways. For in situ generation of Cu(I), 100 μM CuSO₄ and 200 μM of tris-(carboxyethyl)phosphine (TCEP) were added to the cells. Alternatively, the Cu(I) ion was added directly to the cells in the form of an aqueous suspension of CuBr. Briefly, 10 μL of a 10 mM suspension of CuBr (99.999% purity, Aldrich) was thoroughly agitated and added to the cells. As discussed in the Results section, the quality of the CuBr is critical for the success of the experiment. All labeling reactions were allowed to continue for 16 h at 4° C. and were stopped by washing the cells with PBS.

### Example 4

### Mass Spectrometric Analysis

Analysis of peptide mixtures by MudPIT was done essentially as described in Graumann et al. [49] using a LCQ-DecaXP electrospray ion trap mass spectrometer (ThermoElectron). Raw mass spectra were filtered and analyzed with 2 to 3, SEQUEST, DTASelect and Contrast algorithms [33, 34] using standard and modification settings to find modified amino acid residues, like AHA in its possible tagged (+107 mass units) or failed-tagged (−5.1 mass units) version or D10-deuterated 1-leucine (+10 mass units).

The current state-of-the-art tandem mass spectrometry approaches, such as MudPIT, couple in-line multidimensional chromatographic separations with continuous acquisition of data from a column effluent, allow collecting tens of thousands of spectra in a single experiment. Raw mass spectral data were filtered and analyzed using 2 to 3, SEQUEST, DTASelect and Contrast algorithms [33, 34], which validate the data and allow for efficient and comprehensive interpretation and comparison with proteomic databases. In addition to the validation parameters set by the algorithms, the incorporation of stable isotope amino acids, for instance a deuterated variant of L-leucine, can serve as another inner validation for newly synthesized proteins [35]. Certain non-limiting exemplary data addressing the above issues are included in the following section.

### Example 5

### Pharmacology

In pharmacology experiments, the following drugs will be applied at the following concentrations: CNQX: 10 μM (Sigma); APV: 50 μM (Sigma); Anisomycin: 40 μM (Sigma); Cycloheximide: 107 μM (Sigma); DHPG: 50 μM (Tocris); NMDA: 50 μM (Sigma); BDNF: 50 ng/ml (Promega); MG132: 50 μM; glutamate/glycine: 100 μM/10 μM (Sigma), KCl: 90 mM (Sigma).

### Example 6

### Use of a Non-canonical Amino Acid AHA to Label and Identify Newly Synthesized Proteins

This example demonstrates the general suitability of the invention for mammalian cells, such as HEK293 cells and dissociated hippocampal neurons. To assess the purification of a target protein, cells were either transfected (HEK293 cells) or infected (dissociated hippocampal neurons) with a construct coding for a destabilized GFP protein. For neuronal infections, the destabilized and myristoylated GFP reporter described in Aakalu et al. [4] is used. Meanwhile, HEK293 cells are transfected with the pd2EGFP-N1 plasmid (Clontech) using PolyFect (Qiagen). The use of GFP enables monitoring protein levels in intact cells using fluorescence, as well as monitoring protein levels in cell lysates using Western blot analysis.

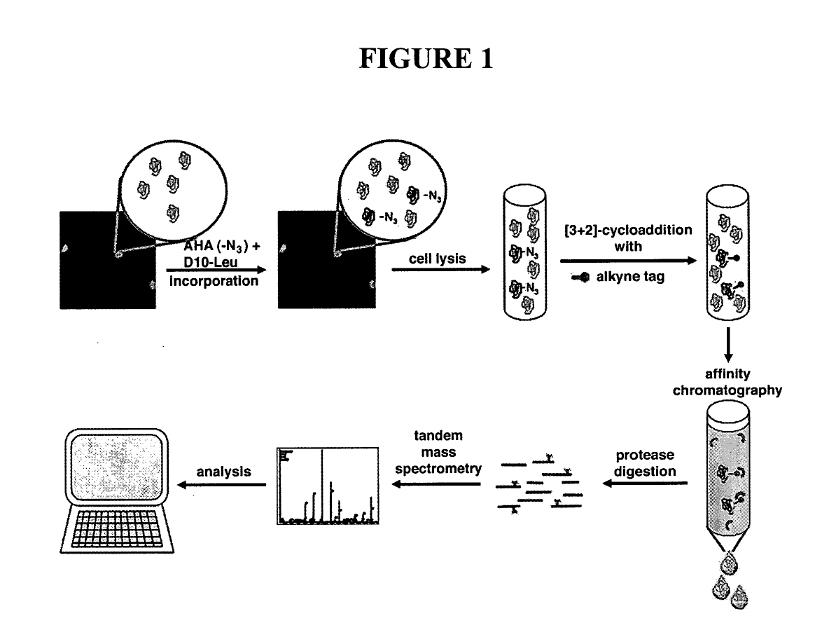

FIG. 1 is a general overview for the suitability of AHA for use in the methods of the invention. Applicants first examined the specificity of AHA incorporation, and its potential toxicity, using whole neuron morphological measurements as well as measurements of protein degradation. Incubation with methionine was used as a general control in all of the performed experiments. In order to visualize their gross morphological structure, neurons were infected with a destabilized form of the fluorescent protein EGFP, and incubated for 1.5 hours with the same molar concentration of either AHA or methionine.

Dissociated hippocampal cultured neurons (12 DIV) were infected with a destabilized and myristoylated variant of GFP, whose mRNA is targeted into dendrites ([4]). Nine hours post infection, cells were incubated for 2 hrs with equimolar concentrations of AHA or methionine. Neurons expressing the GFP reporter indicating no change in the gross morphology of AHA-incubated neurons compared to methionine controls. Thus AHA is not toxic to neurons, as evidenced by intact neuronal processes and continuous non-blebby expression of GFP throughout the dendritic arbor.

Applicants also confirmed the successful incorporation of AHA into proteins in these cultures by subsequent biotinylation with the alkyne linker and Western blot analysis. Expressed GFP was selectively enriched by avidin chromatography from AHA-treated cells as compared to methionine-treated cells. A Western blot of avidin-purified samples was performed using either anti-biotin-antibody or an anti-GFP antibody. In both experiments, the eluate was enriched for the presence of biotin-labeled proteins, indicating that the purification was successful.

To show the specificity of this technique for the detection of newly synthesized proteins, HEK293 cells were incubated for 2 hrs with AHA in the presence or absence of a protein synthesis inhibitor (Anisomycin or Cycloheximide). After incubation, cells were lysed and subjected to [3+2]cycloaddition with the biotin-bearing alkyne-linker Biotin-PEO-Propargylamide [3], followed by Western blot analysis with an anti-Biotin-antibody. No signal was detected in either the methionine or the protein synthesis inhibitor lanes, indicating the specificity of this technique as well as the membrane permeability of the reagent. The potential for increased protein degradation was measured by examining the ubiquitin signal strength on a Western blot from whole lysates of HEK293 cells, which were treated with AHA, methionine or incubation buffer for two hours. No increased ubiquitination was observed in AHA treated cells compared to buffer or methionine controls, indicating that the modified amino acid does not cause severe protein misfolding.

### Example 7

### Purification of AHA-labeled (Biotinylated) Proteins

Following protein synthesis, cell lysates were subjected to a [3+2]-cycloaddition reaction with a biotin-bearing alkyne linker. To initially address our ability to purify AHA-labeled proteins, we have conducted avidin-affinity chromatography of biotinylated GFP, which was expressed in HEK293 cells or neuronal cultures.

In the same system, biotinylated GFP was detected in the eluate of a GFP-immunoprecipitation from AHA-treated cells followed by cycloaddition with the biotin-alkyne tag, but was not detected in methionine-treated control cells. Biotinylated GFP can be immunoprecipitated with a GFP-antibody from AHA-treated neuronal cultures or HEK293 cells, but not from methionine treated cultures.

### Example 8

### Mass Spectrometry

About 200 proteins were identified and validated in a tandem mass spectrometry analysis of single avidin-purified proteins from AHA-treated whole cell lysates of HEK293 cells. In this experiment, identifications for a protein were considered as valid, if at least two fully tryptic peptides were assigned to a protein, with at least one peptide harboring a modification due to AHA or a deuterated leucine.

### Example 9

### An Affinity Reagent with Two Affinity Groups

To increase the specificity of the initial purification of tagged proteins by a two-step procedure. Applicants have developed an affinity reagent which allows for a two-step purification. One goal is to diminish contamination by proteins that adhere non-specifically to the matrices used for affinity-purification. In addition, a tag is introduced, which tag is suitable for detection by mass spectral analysis, and therefore can be used as an inner validation of the data.

The first alkyne tag used in the development of the technique, Biotin-PEO-Propargylamide, resulted in a gain of 578 mass units per reaction and was difficult to detect in the analysis because of its high molecular weight. To increase the purification of the modified proteins and facilitate detection by mass spectrometry, we have designed a new alkyne tag (FIG. 2A) with the following properties: A biotin moiety (Biocytin, for avidin affinity purification) at the N-terminus followed by the FLAG-antibody epitope (DYKDDDDK, SEQ ID NO: 1), which is covalently linked to propargylglycine harboring the reactive alkyne group. A short spacer (GGA) was introduced between Biocytin and the FLAG epitope to ensure the steric accessibility of both biotin and FLAG during affinity purification. This tag can be also proteolytically cleaved by trypsin; following cleavage the resulting mass gain of tagged AHA is 107, which can be easily detected in the mass spectral analysis. Tandem purification is achieved by using first a monomeric avidin resin (Pierce) and then an anti-FLAG-M2 affinity gel (Sigma). In the first experiments, Applicants were able to demonstrate the successful use of this new tag. FIG. 2B shows the subsequent purification of AHA-labeled proteins. Following tagging with the Biotin-FLAG-alkyne tag, AHA-labeled proteins from a HEK293 cell lysate were first subjected to an avidin column. After SDS elution from the matrix and removal of the majority of SDS, these proteins were further purified using an anti-FLAG-M2 affinity gel. Matrix-bound proteins were eluted by boiling in SDS sample buffer. As a control, a cell lysate from methionine-incubated HEK293 cells was treated in parallel. Note the absence of biotin signal in the methionine control. Both matrices allow for specific and mild elution of bound proteins by competition with biotin or FLAG-peptide, respectively, which will increase the specificity of the procedure even more. An establishment of these mild elution conditions is currently underway.

To assess the efficiency of the tandem purification procedure further, Applicants examine the composition of a tandem purified mixture of two proteins, GST and DHFR. One of them is expressed in E. Coli in the presence of AHA in minimal medium and the other is expressed in rich medium containing methionine. After [3+2]-cycloaddition with the tandem featured tag, varying ratios of the two proteins are purified together and analyzed by mass spectrometry for contamination by the non-tagged protein. Additionally, different amounts of a single, tagged protein (e.g., GST) are added to a non-tagged cell lysate. Tandem purification and mass spectrometry are performed to establish how much of a tagged protein is needed for successful identification. Finally, the levels of AHA labeling is varied and determined in order to optimize and characterize the method.

Further internal validation of the mass spectral data is achieved by using 10-fold deuterated L-leucine (D10-L-leucine) together with AHA during protein synthesis [35]. Peptides derived from proteins labeled with AHA+D10-L-leucine are expected to show an increase of 10 mass units per incorporated D10-L-leucine, and as such can be distinguished from peptides derived from preexisting proteins.

### Example 10

### Use of a Non-canonical Amino Acid AHA to Label and Identify Newly Synthesized Proteins in Rat Tissues

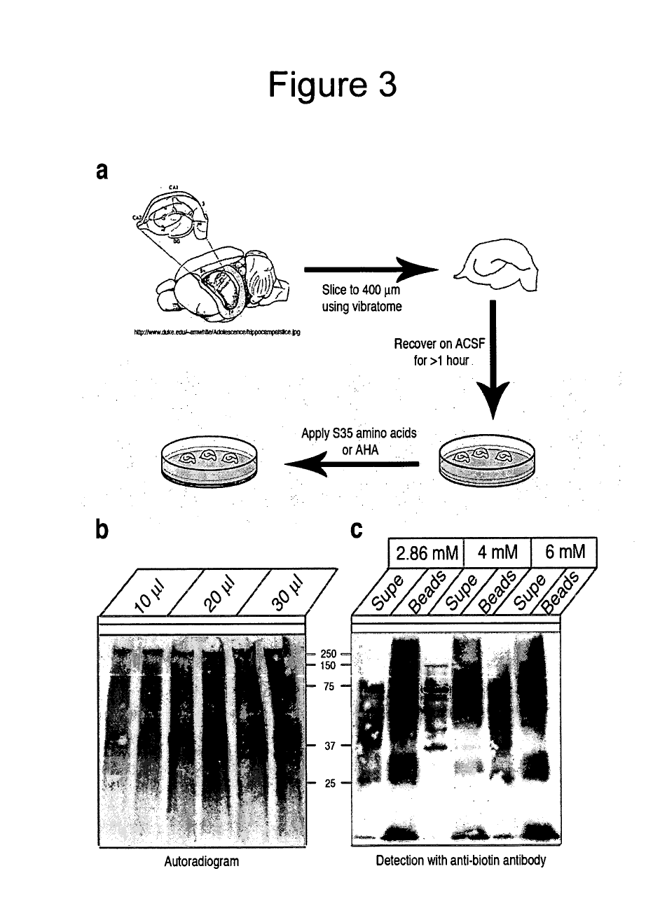

This example demonstrates the general suitability of the invention for use in mammalian tissues, such as in vivo use in a patient. Specifically, FIG. 3 shows an experiment where non-natural amino acid AHA can be successfully incorporated into newly synthesized proteins in sliced rat brain tissue that has maintained its microarchitecture. (A) The method of AHA incorporation in acute hippocampal slices.

In FIG. 3A, about 400 μm-thick rat hippocampal slices were isolated from male Sprague-Dawley rats. These slices were recovered in artificial cerebrospinal fluid (ACSF) for at least 1 hour on organotypic membranes. Upon recovery, the slices were incubated with either a mixture of ³⁵S Met and ³⁵S Cys, or varying concentrations of AHA for 1.5 hours.

FIG. 3B shows autoradiogram of ³⁵S Met and ³⁵S Cys-labeled proteins from acute hippocampal slices. Labeled slices were homogenized and varying volumes of the homogenate are shown here. It is apparent that numerous newly synthesized proteins (smears on the gel) within the slices readily acquire the ³⁵S-labeled amino acids.

FIG. 3C is a Western blot analysis for AHA-incorporated proteins from acute hippocampal slices. After AHA incorporation and slice homogenization, the homogenates were subjected to [3+2]-cycloaddition with a Biotin-PEO tag, and proteins incorporating AHA (and thus subsequently labeled by the Biotin-PEO labeling moiety) were concentrated using Neutravidin-conjugated beads (which binds the Biotin-PEO labeling moiety). Note the vast enrichment of biotinylated proteins due to the incorporation of AHA into the newly synthesized proteins in the acute hippocampal slices.

This experiment demonstrates that the subject labeling reagent (such as AHA) can be used in vivo, since cells in live tissues that have retained the microarchitecture of the organ (brain in this case) can readily intake the labeling reagent in the solution (which is analogous to the labeling reagent in the blood), and incorporate the labeling reagent into numerous newly synthesized proteins using the endogenous protein synthesis machinery of the cells.

### Example 11

### Detection of Increased Protein Synthesis in a Human Hippocampal Dysembryoplastic Neuroepithelial Tumor

Applicants demonstrate here the extension of the labeling of newly synthesized proteins in mammalian cell lines to mammalian tissues (such as brain), and thus its applicability in whole organisms.

While important and invaluable information has been obtained in recent years from both untransformed and transformed tumor cell lines, these systems lack the ability to capture early stages of the transformation process, and are limited to available, i.e. easily cultivatable cell lines. Moreover, a given cell line might not provide an accurate picture of cellular processes, since it must adapt to the environment of the cell culture system.

In order to identify dynamic proteomic changes of more demanding cell lines and tissue, rapid labeling in intact tissues and cells is needed. Our previous experiments have shown that AHA behaves like a natural nutrient with excellent membrane permeability and undetected cell toxicity, thereby allowing short labeling times for experiments of the kind described above. Here, Applicants explore the use of the subject methods and systems in acutely dissected mammalian tissues to identify newly synthesized proteins from both normal and diseased human tissue.

In the first series of experiments, Applicants explored proper conditions for labeling freshly dissected rat tissues with AHA, by dissecting healthy rat brain tissues, and splitting the dissected tissue into two sets. One set was kept on ice in HBS (HEPES-buffered saline) buffer, the other was flash-frozen in liquid nitrogen (N₂).

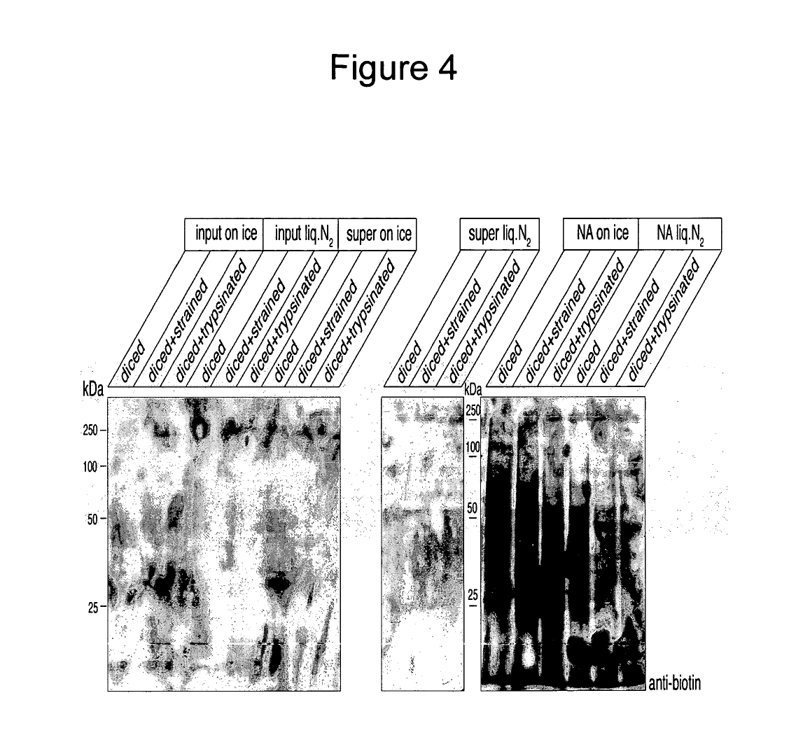

For each set, the brain tissues were either (1) diced, (2) diced and strained, or (3) diced, trypsinized and triturated, before incubation in AHA for 1 hour. The tissues were then lysed in SDS and Tx-100, and the lysates were subject to Neutravidin (NA) purification. To normalize the samples, equal total protein was loaded onto the gel for each sample treatment. The results were shown in FIG. 4.

It is apparent in FIG. 4 that different sample treatments (e.g., on ice or flash-frozen in liquid nitrogen) do not appear to have a significant effect on AHA incorporation into the dissected tissues. However, in all treatments, NA substantially enriched newly synthesized proteins that have incorporated AHA (compare the lanes labeled “NA on ice” or “NA liquid N₂” with the lanes labeled “super(natant)” or “input”).

Having obtained the proper conditions for labeling dissected tissues, Applicants repeated the experiments in freshly dissected human DNT tumor tissues and control tissues. Specifically, as a first demonstration of this approach, proteomic profiling of tissue of a female human patient suffering from a hippocampal dysembryoplastic neuroepithel tumor (DNT) was performed using the subject method. As a control, non-diseased tissue (lateral temporal cortex) from the same patient was analyzed along with the DNT tissue.

A DNT is similar in behavior to an oligodendroglioma. Although it occurs both in adults and children, the average patient with this Grade I, slow-growing tumor is under the age of twenty, and has a history of uncontrollable seizures of the partial complex type. A DNT is most commonly located in a temporal or frontal lobe of the cerebrum. A DNT is usually diagnosed following a long history of seizures. Surgery alone often results in long-term control for this tumor.

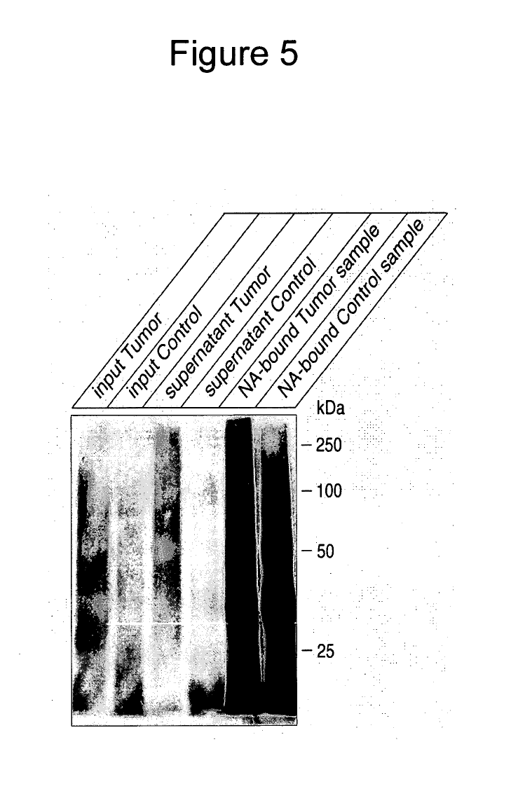

Freshly dissected tumor and control tissue were obtained in operating room. To ensure optimal uptake of AHA, both tissues were trypsinized and triturated into single cells before treatment with AHA and deuterated 1-lysine. Incubation with AHA was done at 37° C. under constant agitation. The tissues were subsequently lysed in SDS and Tx-100 buffer, and the newly synthesized proteins were Neutravidin (NA)-purified. The tagging and purification steps were done as described in Dieterich et al., Proc Natl Acad Sci USA 103: 9482-9487, 2006 (incorporated by reference). Equal total protein were used as input for each gel lane in FIG. 5.

It is apparent in FIG. 5 that NA-purification vastly enriched the newly synthesized proteins that have incorporated AHA. Compared to normal tissue, even the slow-growing tumor tissue was differentially labeled, in that levels of protein synthesis were substantially higher in cells from tumor tissue compared to non-diseased tissue, which was demonstrated by intensive anti-biotin signal in the Neutravidin-bound tumor sample lane (compare the much darker smear on the tumor lane with that of the normal tissue lane where discrete protein bands can still be seen).

Thus Applicants have demonstrated in this experiment that even in slow growing tumor, where new protein synthesis is not as robust as in other more aggressively-growing tumors, the subject labeling reagent (e.g., AHA) can differentially label tumor cells (e.g., weak hippocampal DNT) as compared to cells from the surrounding normal tissue (e.g., lateral temporal cortex).

In addition, to confirm that more active synthesis of proteins in tumor tissue compared to normal tissues, Applicants performed on-resin trypsination of the purified proteins from both samples, and subject them to mass spectrometrical (MS) analysis using tandem MS on LTQ.

Specifically, for MS analysis, the Neutravidin-bound samples were digested and subjected to tandem MS as described previously (Dieterich et al., Proc Natl Acad Sci USA 103: 9482-9487, 2006, incorporated by reference). In this experiment a total of 503 proteins were identified in the tumor sample only, 131 identifications were shared by the tumor and the control sample and 22 proteins were identified in the control only. Among those 503 proteins of the tumor sample, 10 identified candidates are related to transcription factor activity (e.g., Prohibitin, see Fusaro et al., J. Biol. Chem. 278: 47853-61, 2003; a breast cancer antigen and TCP4, see Banerjee et al., Mol. Cell. Biol. 24: 2052-62, 2004; a p53 function activator), and 9 candidates are involved in cell cycle regulation (e.g., Septins 2, 4 and 9, see Kim et al., Neoplasia 6:168-78, 2004).

Taken together these data show that the subject system and methods can be used to determine the proteome of rapidly dividing cells, such as tumor cells, both in vitro and in vivo. The method relies on the bioorthogonal incorporation of non-canonical amino acid into newly synthesized proteins and the subsequent detection of the reactive group of the non-canonical amino acid with a suitable custom-designed coupling reagent bearing moieties for affinity-purification, visualization or both. In the example described above, azide-bearing newly synthesized proteins were detected with an alkyne affinity tag.

### Example 12

### Use of Exemplary Non-canonical Amino Acids to Label and Identify Newly Synthesized Proteins in Dissociated Neurons

This set of experiments describe, inter alia, the double-labeling of cells (e.g., neurons) with two pairs of different non-natural amino acid/labeling reagents, the time course of labeling, and local perfusion of non-natural amino acids to sections of neuron dendrites.

In certain embodiments of the invention, two or more pairs of different non-natural amino acid/labeling reagents may be used to simultaneously or sequentially label the newly synthesized proteins. For example, FIG. 6A shows the structures of the two exemplary pairs AHA-TRA-tag and HPG-FLA-tag. FIG. 6B shows the result of using these pairs separately in dissociated hippocampal neurons (division 17, or “DIV” 17). The DIV 17 neurons were incubated in either 4 mM AHA, 4 mM methionine (Met), or 4 mM AHA and 40 μM anisomycin (Aniso) for 1 hour, and then tagged with 1 μM TRA or FLA tag, and immunostained for the dendritic marker protein MAP2. See the 4 panels at the left side of FIG. 6B (scale bar=20 μm). The experiment demonstrated that AHA was successfully incorporated in the dissociated hippocampal neurons under the experimental conditions. Such incorporation depends on new protein synthesis, since protein synthesis inhibitor Anisomycin completely abolished AHA incorporation.

Similarly, HPG can be successfully incorporated into the same neurons, in a new protein synthesis-dependent manner (see the right 4 panels in FIG. 6B).

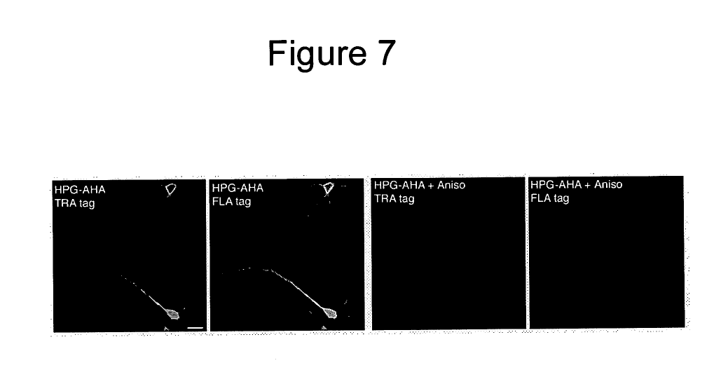

FIG. 7 (scale bar=20 μm) shows that both pairs may be used sequentially in the same cells, and separately detected afterwards. Specifically, dissociated hippocampal neurons (DIV 16) were incubated in 4 mM AHA for 1.5 hrs, followed by 4 mM HPG for 1.5 hrs, or in the presence of 40 μM anisomycin (Aniso). The cells were then sequentially tagged with 1 μM TRA and 1 μM FLA tag for 12 hours each. The results indicate that both labelling reagents can be separately detected in the double-labelling experiments.

Although labelling reagents with different colors were used in this experiment, other embodiments, such as using one labelling reagent with fluorescent tag with one labelling reagent with radioactive tag, are readily apparent to a skilled artisan.

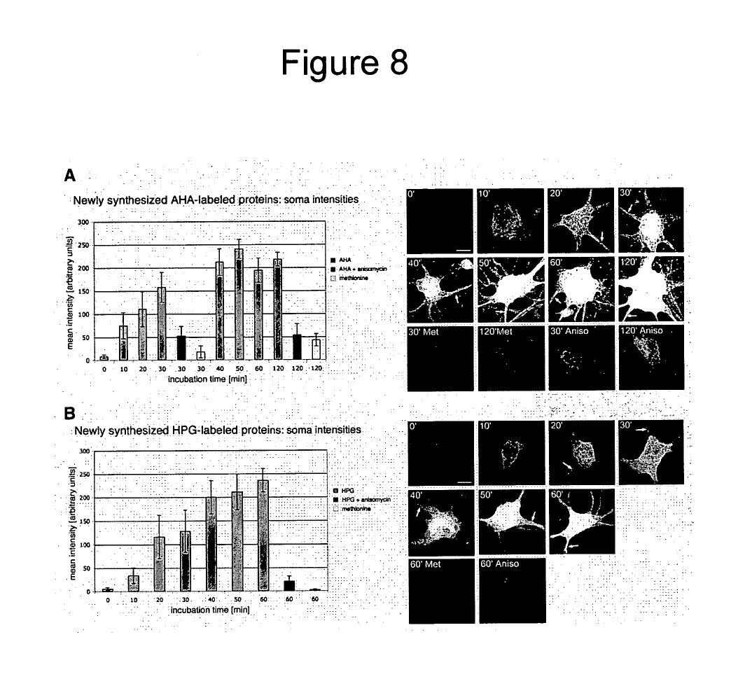

FIG. 8 shows the time-course of protein synthesis in the somata of hippocampal neurons after AHA—(FIG. 8A) or HPG (FIG. 8B) incorporation and [3+2] cycloaddition with fluorescent tags. Cultures were incubated for time points indicated with 4 mM AHA (FIG. 8A), 4 mM methionine (Met) or 4 mM AHA in the presence of 40 μM anisomycin (Aniso) or HPG (FIG. 8B). After cycloaddition with the fluorescent TRA tag (FIG. 8A) or FLA tag (FIG. 8B), images were taken with same acquisition parameters on a Zeiss meta 510 confocal microscope. Graph represents mean intensities with standard deviation of the somata. Per time point data of 20-50 cells were collected. Representative samples are shown in the right panel. Arrows point to dendritic processes. Scale bar=5 μm.

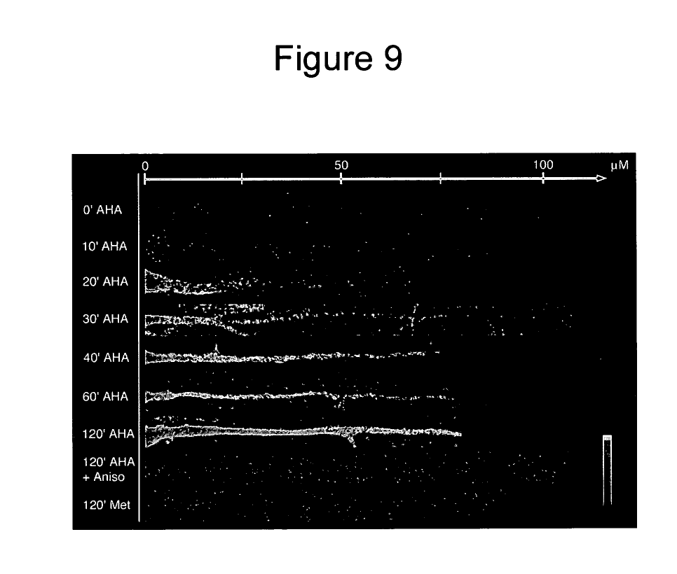

FIG. 9 shows the time-course for the detection of newly synthesized proteins in dendrites of hippocampal neurons. Representative straightened dendrites of neurons (DIV 16) incubated with 4 mM AHA, 4 mM Methionine (Met) or AHA plus 40 μM anisomycin (Aniso) for time points indicated. The lookup table indicates fluorescence intensity. The left end is the proximal end, the right end is the distal end. It is apparent that AHA incorporation was visible after 10 minutes. It became obvious at the proximal end by 20 minutes, and there was substantial diffusion to the distal end by 30 min. And by 40 min., the whole neuron soma and dendrite was filled with AHA-labeled proteins. This process was completely inhibited at the presence of protein synthesis inhibitor anisomycin, and there is essentially no background (see the Met control).

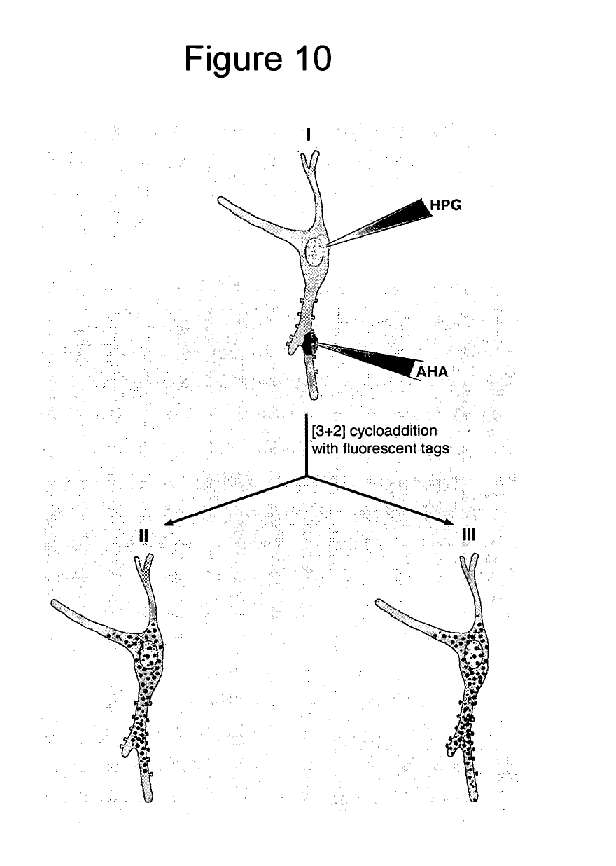

FIG. 10 is a schematic drawing showing a strategy to use double-labeling to visualize endogenous newly synthesized proteins in soma and dendrites of hippocampal neurons. Soma and a single dendrite of a neuron can be locally microperfused with the two different non-canonical amino acids, for example, AHA and HPG (I). AHA- or HPG-incorporated proteins will then be detected with two specific fluorescent tags (e.g., Sulforhodamine and Fluorescein) via [3+2] cycloaddition. Two scenarios are shown for which normal (II) and abnormal (III) synaptic function alters the contribution of somatically and dendritically newly synthesized proteins.

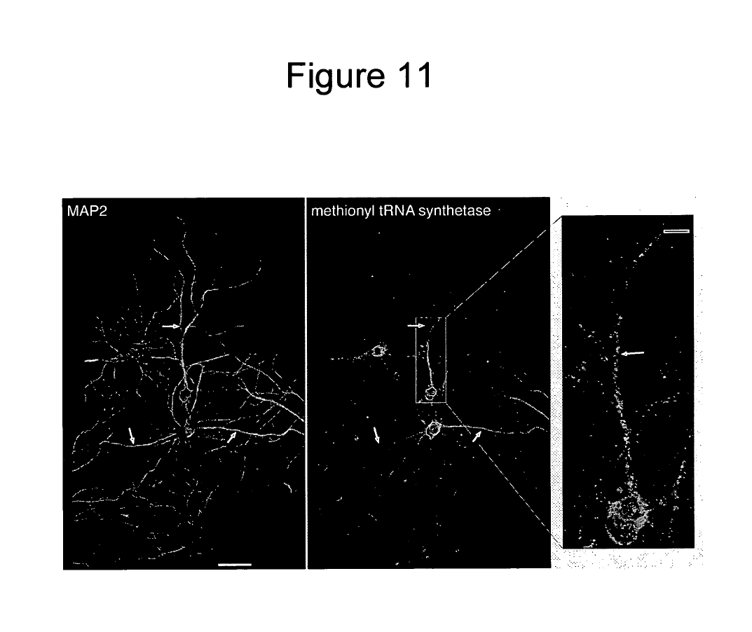

FIG. 11 shows immunohistochemical detection of dissociated hippocampal neurons (DIV 17) for the dendritic marker protein MAP2 and methionyl tRNA synthetase. Arrows indicate dendritic processes. Scale bar is 50 μm in the overview, and 10 μm in the close-up.

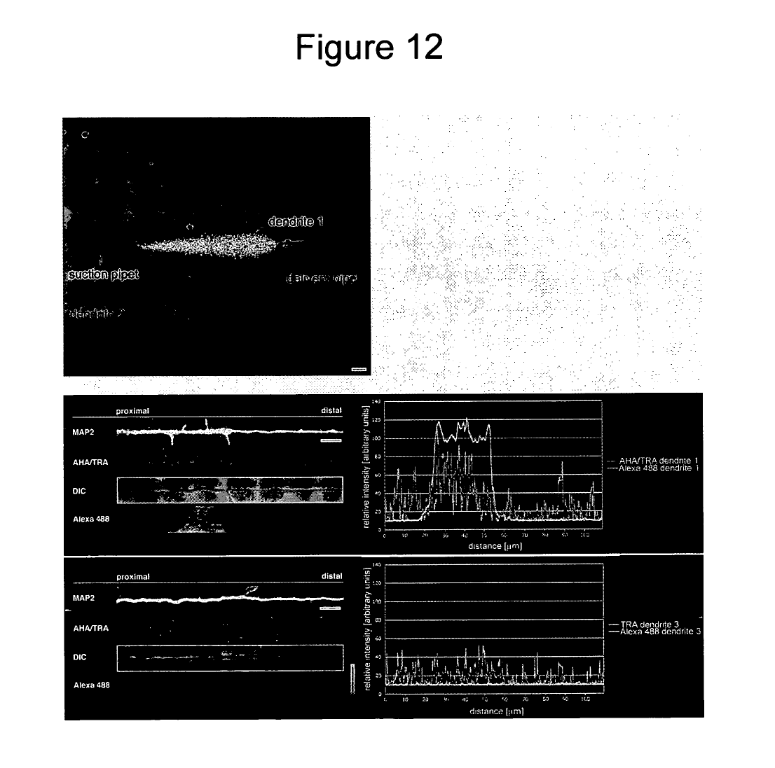

FIG. 12 (scale bars=10 μm) shows the result of local perfusion of dendrites with AHA. Specifically, a dissociated hippocampal neuron (DIV 20) was locally perfused with 4 mM AHA for 75 min. Size of the perfusion spot was monitored using Alexa488 in the perfusion solution. After fixation and subsequent tagging with 1 μM TRA tag, an immunostaining was performed for the dendritic marker protein MAP2. Dendrites were straightened with the ImageJ software. The same technique may be used to infuse a second non-natural amino acid (such as HPG) into the soma and shown in FIG. 10.

### REFERENCES

1. Zhang et al., Chemical probes and tandem mass spectrometry: a strategy for the quantitative analysis of proteomes and subproteomes. Curr Opin Chem Biol 8(1): 66-75, 2004.
2. Kiick et al., Incorporation of azides into recombinant proteins for chemoselective modification by the Staudinger ligation. Proc Natl Acad Sci USA 99(1): 19-24, 2002.
3. Link and Tirrell, Cell Surface Labeling of Escherichia coli via Copper(I)-Catalyzed [3+1] Cycloaddition. Journal of the American Chemical Society 125(37): 11164-11165, 2003.
4. Aakalu et al., Dynamic visualization of local protein synthesis in hippocampal neurons. Neuron 30(2): 489-502, 2001.
5. Huber et al., Role for rapid dendritec protein synthesis in hippocampal mGluR-dependent long-term depression. Science 288(5469): 1254-7, 2000.
6. Kang and Schuman, A requirement for local protein synthesis in neurotrophin-induced hippocampal synaptic plasticity. Science 273(5280): 1402-6, 1996.
7. Kacharmina et al., Stimulation of glutamate receptor protein synthesis and membrane insertion within isolated neuronal dendrites. Proc Natl Acad Sci USA 97(21): 11545-50, 2000.
8. Ouyang et al., Visualization of the distribution of autophosphorylated calcium/calmodulin-dependent protein kinase II after tetanic stimulation in the CAI area of the hippocampas. J Neurosci 17(14): 541-627, 1997.
9. Ouyang et al., Tetanic stimulation leads to increased accumulation of Ca²⁺/calmodulin-dependent protein kinase H via dendritic protein synthesis in hippocampal neurons. J Neurosci. 19(18): 7823-33, 1999.
10. Steward and Levy, Preferential localization of polyribosomes under the base of dendritic spines in granule cells of the dentate gyrus. J Neurosci 2(3): 284-91, 1982.
11. Steward and Fass, Polyribosomes associated with dendritic spines in the denervated dentate gyrus: evidence for local regulation of protein synthesis during reinnervation. Prog Brain Res 58: 131-6, 1983.
12. Steward and Falk, Protein-synthetic machinery at postsynaptic sites during synaptogenesis: a quantitative study of the association between polyribosomes and developing synapses. J Neurosci 6(2): 412-23, 1986.
13. Nguyen et al., Requirement of a critical period of transcription for induction of a late phase of LTP. Science 265(5175): 1104-7, 1994.
14. Martin et al., Synapse-specific, long-term facilitation of aplysia sensory to motor synapses: a function for local protein synthesis in memory storage. Cell 91(7): 927-38, 1997.
15. Kang and Schuman, A requirement for local protein synthesis in neurotrophin-induced hippocampal synaptic plasticity. Science 273(5280): 1402-6, 1996.
16. Casadio et al., A transient, neuron-wide form of CREB-mediated long-term facilitation can be stabilized at specific synapses by local protein synthesis. Cell 99(2): 221-37, 1999.
17. Bito et al., CREB phosphorylation and dephosphorylation: a Ca²⁺— and stimulus duration-dependent switch for hippocampal gene expression. Cell 87(7): 1203-14, 1996.
18. Steward and Schuman, Protein synthesis at synaptic sites on dendrites. Annu Rev Neurosci 24: 299-325, 2001.
19. Steward and Schuman, Compartmentalized synthesis and degradation of proteins in neurons. Neuron 40(2): 347-59, 2003.
20. Wu et al., CPEB-mediated cytoplasmic polyadenylation and the regulation of experience-dependent translation of alpha-CaMKII mRNA at synapses. Neuron 21(5): 1129-39, 1998.
21. Weiler et al., Synapse-activated protein synthesis as a possible mechanism of plastic neural change. Prog Brain Res 100: 189-94, 1994.
22. Steward, mRNA localization in neurons: a multipurpose mechanism? Neuron 18(1): 9-12, 1997.
23. Torre and Steward, Demonstration of local protein synthesis within dendrites using a new cell culture system that permits the isolation of living axons and dendrites from their cell bodies. J Neurosci 12(3): 762-72, 1992.
24. Glanzer and Eberwine, Mechanisms of translational control in dendrites. Neurobiol Aging 24(8): 1105-11, 2003.
25. Crino and Eberwine, Molecular characterization of the dendritic growth cone: regulated mRNA transport and local protein synthesis. Neuron 17(6): 1173-87, 1996.
26. Crino et al., Presence and phosphorylation of transcription factors in developing dendrites. Proc Natl Acad Sci USA 95(5): 2313-8, 1998.
27. Job and Eberwine, Identification of sites for exponential translation in living dendrites. Proc Natl Acad Sci USA 98(23): 13037-42, 2001.
28. Ju et al., Activity-dependent regulation of dendritic synthesis and trafficking of AMPA receptors. Nat Neurosci 7(3): 244-53, 2004.
29. Brendel et al., Characterization of Staufen 1 ribonucleoprotein complexes. Biochem J 384(Pt 2): 239-46, 2004.
30. Eberwine et al., Local translation of classes of mRNAs that are targeted to neuronal dendrites. Proc Natl Acad Sci USA 98(13): 7080-5, 2001.
31. Gygi et al., Quantitative analysis of complex protein mixtures using isotope-coded affinity tags. Nat Biotechnol 17(10): 994-9, 1999.
32. Washburn et al., Large-scale analysis of the yeast proteome by multidimensional protein identification technology. Nat Biotechnol 19(3): 242-7, 2001.
33. Tabb et al., DTASelect and Contrast: tools for assembling and comparing protein identifications from shotgun proteomics. J Proteome Res 1(1): p. 21-6, 2002.
34. Ducret et al., High throughput protein characterization by automated reverse-phase chromatography/electrospray tandem mass spectrometry. Protein Sci 7(3): 706-19, 1998.
35. Ong et al., Stable isotope labeling by amino acids in cell culture, SILAC, as a simple and accurate approach to expression proteomics. Mol Cell Proteomics 1(5): 376-86, 2002.
36. Bassell et al., Sorting of beta-actin mRNA and protein to neurites and growth cones in culture. J Neurosci 18(1): 251-65, 1998.
37. Kiebler et al., Purification and characterization of rat hippocampal CA3-dendritec spines associated with mossy fiber terminals. FEBS Lett 445(1): 80-6, 1999.
38. Meffert et al., Nitric oxide stimulates Ca²⁺-independent synaptic vesicle release. Neuron 12(6): 1235-44, 1994.
39. Torre and Steward, Protein synthesis within dendrites: glycosylation of newly synthesized proteins in dendrites of hippocampal neurons in culture. J Neurosci 16(19): 5967-78, 1996.
40. Gerber et al., Absolute quantification of proteins and phosphoproteins from cell lysates by tandem MS. Proc Natl Acad Sci USA 100(12): 6940-5, 2003.
41. Peng et al., Semi-quantitative proteomic analysis of rat forebrain postsynaptic density fractions by mass spectrometry. J Biol Chem 279(20): 21003-11, 2004.
42. Yin et al., The brain-derived neurotrophic factor enhances synthesis of Arc in synaptoneurosomes. Proc Natl Acad Sci USA 99(4): 2368-73, 2002.
43. Scbratt et al., BDNF regulates the translation of a select group of mRNAs by a mammalian target of rapamycine-phosphatidylinositol 3-kinase-dependent pathway during neuronal development. J Neurosci 24(33): 7366-77, 2004.
44. Bagni et al., Chemical stimulation of synaptosomes modulates alpha-Ca²⁺/calmodulin-dependent protein kinase II mRNA association to polysomes. J Neurosci 20(10): RC76, 2000.
45. Agard et al., A strain promoted [3+2] azide-alkyne cycloaddition for covalent modification of biomolecules in living systems. J Am Chem Soc 126(46): 15046-7, 2004.
46. Mangold et al., Azidoalanine mutagenicity in Salmonella: effect of homologation and alpha-methyl substitution. Mutat Res 216(1): 27-33, 1989.
47. Wang et al., Bioconjugation by copper(I)-catalyzed azide-alkyne [3+2] cycloaddition. J Am Chem Soc 125(11): 3192-3, 2003.
48. Malgaroli and Tsien, Glutamate-induced long-term potentiation of the frequency of miniature synaptic currents in cultured hippocampal neurons. Nature 357(6374): 134-9, 1992.
49. Graumann et al., Applicability of tandem affinity purification MudPIT to pathway proteomics in yeast. Mol Cell Proteomics 3(3): 226-37, 2004.

The contents of all cited references (including literature references, issued patents, published patent applications as cited throughout this application) are hereby expressly incorporated by reference. The entire teaching of U.S. Ser. No. 11/314,323, and all of its priority applications are hereby expressly incorporated by reference.

### EQUIVALENTS

Those skilled in the art will recognize, or be able to ascertain using no more than routine experimentation, numerous equivalents to the specific method and reagents described herein, including alternatives, variants, additions, deletions, modifications and substitutions. Such equivalents are considered to be within the scope of this invention and are covered by the following claims.

1. A method for detecting or treating cancer in a patient, comprising administering to the patient a pharmaceutical composition comprising:
(1) a non-natural amino acid comprising a first reactive group;
(2) a labeling reagent comprising a second reactive group and a labeling moiety;
under conditions wherein the non-natural amino acid is preferentially incorporated into the newly synthesized proteins of the cancer;
wherein the first and second reactive groups react to label the non-natural amino acid with the labeling reagent;
wherein the labeling reagent comprises a detectable label, inhibits the progression of the cancer, and/or facilitates the killing of the cancer.

2. (canceled)

3. The method of claim 1, wherein the first reactive group is an azido group.

4. The method of claim 3, wherein the non-natural amino acid is azidoalanine, azidohomoalanine (AHA), azidonorvaline, or azidonorleucine.

5. The method of claim 1, wherein the first reactive group is a ketone or aldehyde moiety, a diboronic acid moiety, or a terminal alkyne moiety.

6. (canceled)

7. The method of claim 1, wherein the labeling reagent comprises a chelate moiety for chelating a metal.

8. The method of claim 7, wherein the labeling reagent is a chelator for a radiometal or a paramagnetic ion.

9. The method of claim 8, further comprising infusing into the patient an effective amount of chelator compounds.

10. The method of claim 9, wherein the chelator compound is EDTA or DTPA.

11. The method of claim 7, wherein the labeling reagent is a chelator for a radionuclide useful for radiotherapy or imaging procedures.

12. The method of claim 11, wherein the radionuclide is a beta- or alpha-emitter for radio-therapeutic use.

13. The method of claim 11, wherein said radionuclide is a gamma-emitter, positron-emitter, Auger electron-emitter, X-ray emitter or fluorescence-emitter.

14. The method of claim 11, wherein said radionuclide is ^{99m}Tc (technium).

15. The method of claim 1, wherein the labeling reagent comprises a bifunctional chelator NₓS_{y} that are capable of coordinately binding a metal or radiometal, wherein x and y are integers between 1 and 4.

16. The method of claim 15, wherein NₓS_{y} has a N₂S₂ or a N₃S core.

17. The method of claim 1, wherein the labeling reagent comprises a cytotoxic moiety.

18. The method of claim 17, wherein said cytotoxic moiety is a radiosensitizing agent, a Boron addend, a chemotherapeutic agent, a protein synthesis inhibitor, a prodrug activated by host metabolism, a cytotoxic toxin, an enzyme that converts prodrug locally, or a dye used in photodynamic therapy or in conjunction with appropriate non-ionizing radiation.

19. The method of claim 18, wherein the radiosensitizing agent is selected from: nitroimidazoles, metronidazole or misonidazole.

20. The method of claim 18, wherein said Boron addend is carborane.

21. The method of claim 18, wherein said chemotherapeutic agent is: taxol; a nitrogen mustard; an ethylenimine derivative; an alkyl sulfonate; a nitrosourea; a triazene; a pyrimidine analog; a purine analog; a vinca alkaloid; an antibiotic; an enzyme; a platinum coordination complex; a substituted urea; a methyl hydrazine derivative; an adrenocortical suppressant; or a hormone and antagonist selected from: an adrenocortisteroid, a progestin selected from hydroxyprogesterone caproate, medroprogesterone acetate or megestrol acetate, an estrogen selected from diethylstilbestrol or ethinyl estradiol, an antiestrogen, or an androgen selected from testosterone propionate or fluoxymesterone.

22. The method of claim 18, wherein said protein synthesis inhibitor is puromycin, cycloheximide, or ribonuclease.

23. The method of claim 18, wherein the labeling reagent comprises a prodrug that is only activated from its inactive precursor form by host metabolism.

24. The method of claim 18, wherein said cytotoxic toxin is selected from: ricin, ricin A chain (ricin toxin), Pseudomonas exotoxin (PE), diphtheria toxin (DT), Clostridium perfringens phospholipase C(PLC), bovine pancreatic ribonuclease (BPR), pokeweed antiviral protein (PAP), abrin, abrin A chain (abrin toxin), cobra venom factor (CVF), gelonin (GEL), saporin (SAP), modeccin, viscumin or volkensin.

25. The method of claim 18, wherein said enzyme that converts prodrug locally is alkaline phosphatase, and said prodrug is etoposidephosphate.

26. The method of claim 17, wherein the cytotoxic moiety is administered to the patient at a dose that contain 10-100 times less active agent as an active moiety than the dosage of agent administered as unconjugated active agents.

27. The method of claim 1, wherein the labeling reagent comprises an antigenic moiety that can be recognized by an antibody.

28. The method of claim 27, wherein the labeling moiety is biotin, and the antigenic moiety is an epitope tag.

29. The method of claim 28, wherein the epitope tag is FLAG tag.

30. The method of claim 29, wherein the FLAG tag comprises one or more cleavage sites for a sequence-specific protease.

31. The method of claim 30, wherein the sequence-specific protease is trypsin.

32. The method of claim 1, wherein the second reactive group and the labeling moiety are linked by one or more cleavable functional groups.

33. The method of claim 32, wherein the cleavable functional groups are photo-cleavable groups, chemically cleavable groups, or enzymatically cleavable groups.

34. The method of claim 1, wherein the second reactive group and the affinity moiety are linked by a photo-cleavable linker.

35. The method of claim 1, further comprising administering to the patient a second pharmaceutical composition comprising:
(3) a second non-natural amino acid comprising a third reactive group;
(4) a second labeling reagent comprising a fourth reactive group and a second labeling moiety;
under conditions where the second non-natural amino acid is preferentially incorporated into the newly synthesized proteins of the cancer;
wherein the third and fourth reactive groups react to label the second non-natural amino acid with the second labeling reagent;
wherein at least one of the labeling reagents inhibits the progression and/or facilitates the killing of the cancer.

36. A high throughput screening method for identifying a compound that inhibits cell proliferation, the method comprising:
(1) contacting a control cell with a non-natural amino acid comprising a first reactive group, under conditions where the non-natural amino acid is preferentially incorporated into the newly synthesized proteins of the control cell;
(2) contacting a control cell with a labeling reagent comprising a second reactive group and a labeling moiety, wherein the first and second reactive groups react to label the non-natural amino acid with the labeling reagent;
(3) repeat steps (1) and (2) to label a test cell at the presence of a candidate compound;
(4) comparing the quantity of the labeling moiety in the control cell and the test cell, respectively;
wherein a decrease in quantity of the labeling moiety in the test cell is indication that the candidate compound inhibits proliferation of the test cell.

37. The method of claim 36, wherein the control cell and the test cell are primary cancer cells from the same cancer, or are from the same cancer cell line.

38. (canceled)

39. The method of claim 36, wherein the labeling moiety is a fluorescent moiety or a reagent that can be subsequently coupled to a fluorescent reagent, and wherein step (4) is effectuated by monitoring fluorescent intensity.

40. A method for detecting or imaging cancer in a patient, comprising:
(a) contacting the patient with a non-natural amino acid comprising a first reactive group, under conditions where the non-natural amino acid is preferentially incorporated into the newly synthesized proteins of the cancer;
(b) contacting the patient with a labeling reagent comprising a second reactive group and a labeling moiety, wherein the first and second reactive groups react to label the non-natural amino acid with the labeling reagent;
(c) detecting the labeling moiety, thereby detecting cancer in the patient.

41. The method of claim 40, wherein the labeling moiety comprises an imaging agent.

42. The method of claim 41, wherein the imaging agent is a radionuclide imaging agent.

43-46. (canceled)
